(19) United States
(12) Patent Application Publication
(10) Pub. No.: US 2025/0122268 A1
(43) Pub. Date: Apr. 17, 2025

(54) COLLAGEN AND USE THEREOF

(71) Applicant: SHANXI JINBO BIO-PHARMACEUTICAL CO., LTD., Taiyuan (CN)

(72) Inventors: Xia Yang, Taiyuan (CN); Xiaobin Lan, Taiyuan (CN); Lingling Wang, Taiyuan (CN); Yongjian Zhang, Taiyuan (CN); Xin Liu, Taiyuan (CN); Zengyao Liu, Taiyuan (CN); Wenfei Yang, Taiyuan (CN)

(21) Appl. No.: 18/933,318

(22) Filed: Oct. 31, 2024

Related U.S. Application Data
(63) Continuation of application No. PCT/CN2023/134277, filed on Nov. 27, 2023.

(30) Foreign Application Priority Data
May 12, 2023 (CN) 202310537499.9

(51) Int. Cl. C07K 14/78; A61K 38/00

(52) U.S. Cl. CPC C07K 14/78; A61K 38/00

(57) ABSTRACT

Provided is collagen and uses thereof. Collagen includes an N-terminal sequence and a C-terminal sequence, wherein the N-terminal sequence includes one or more repeating units, and the repeating unit includes an amino acid sequence shown in SEQ ID NO. 15. The recombinant type II humanized collagen prepared by the present invention has high activity in promoting cell adhesion, does not produce an immune response when applied to the human body, can be obtained on a large scale, and is expected to be widely used in the field of cartilage repair.

### CROSS-REFERENCE TO RELATED APPLICATIONS

This application is a continuation of International Patent Application No. PCT/CN2023/134277 filed on Nov. 27, 2023 which claims the priority benefit of the Chinese Patent Application No. 202310537499.9, filed on May 12, 2023, titled “POLYPEPTIDE AND USE THEREOF”. The content of the aforementioned application is incorporated herein by reference.

### SEQUENCE LISTING

A sequence listing contained in the file named “C23W5040.01US.TCA” which is 46,309 bytes and created on Sep. 26, 2024, is filed electronically herewith and incorporated by reference in its entirety.

### TECHNICAL FIELD

The present disclosure belongs to the field of synthetic biotechnology, and specifically relates to human body structural materials and biosynthetic preparation methods thereof.

### BACKGROUND OF THE INVENTION

Collagen is a type of protein widely distributed in human connective tissue and is also the most abundant protein in the human body, accounting for 25% to 35% of the total protein. It has been found that there are at least 28 collagen subtypes in the human body, which are located in different tissues and organs.

Type II collagen, a high-molecular protein, is mainly found in cartilage tissue, the vitreous body, and cornea, the filamentous collagen fiber of which is interwoven with elastin and polysaccharide proteins to form a network structure, also known as complex bone collagen. Type II collagen is an essential component for cartilage and bone formation, bone growth, and maintenance of mature cartilage, so non-denatured collagen can be used as a structural and functional component of the cartilage.

Type II collagen, as the main component of the articular cartilage matrix, together with lubricating components such as hyaluronic acid and proteoglycans, protects the cartilage from wear and tear. As age increases, the synthesis rate of collagen gradually decreases, the loss of collagen intensifies, articular cartilage degenerates, and the friction between bones intensifies, which can trigger inflammation in the joints. At present, the common treatment method for osteoarthritis is to consume collagen, but its protein utilization rate is low, resulting in slow cartilage regeneration, which is not beneficial to the patient's recovery and affects the process and efficiency of the treatment. Due to the unsatisfactory efficacy of current treatment methods, patients often have to choose artificial joint replacement, which is not only expensive, but may also lead to catastrophic complications such as postoperative thrombosis, and infection after replacement leading to amputation.

However, the collagen material currently used as the cartilage is mainly from animal cartilage extraction. The immunogenicity of animal-origin collagen cannot be eliminated and the impurity removal and extraction processes thereof are still relatively complicated, thus, it is difficult to ensure the triple helix structure of collagen and cannot be used for large-scale production.

At the same time, the animal-origin immune response is also an important reason that limits the application of collagen. With the growing growth of our country's collagen industry, the use of biosynthetic pathways to obtain collagen has become increasingly mature, especially humanized collagen, which has been at the forefront of the world. In 2021, the National Medical Products Administration made a naming and classification of the biosynthetic collagen, wherein the recombinant humanized collagen refers to the full-length or partial amino acid sequence functional region encoded by the specific type of the human collagen gene prepared by DNA recombinant technology, or the combination of the functional regions containing the human collagen function.

The traditional method of producing the collagen is to use acid, alkali, and enzymatic hydrolysis to process the animal source tissues and extract the collagen derivatives. The collagen extracted by these methods has lost its original biological activity and thus cannot be applied in the biomedical field to play a real role. With the development of modern technology, some extraction methods of impurity removal and enzymatic extraction for animal cartilage to obtain the non-denatured type II collagen have emerged at home and abroad. Although the collagen prepared by these methods can be used for cartilage repair, the time for impurity removal and extraction is long, the product purity is low, and the product stability is poor, so it is not beneficial to large-scale production. Given the defects of the existing technology, some companies have proposed using the Pichia pastoris as a host strain to prepare the type II collagen for cartilage repair. However, this collagen is not humanized collagen and therefore has a certain immunogenic response.

Therefore, there is an urgent need for a recombinant type II humanized collagen that can be directly injected into the human body and does not cause an immunogenic response, to be used as a human body structural material applied for cartilage repair.

### SUMMARY OF THE INVENTION

Human body structural materials are mainly structural proteins including collagen, which have complex structures and precise function and are of great significance for the repair and regeneration of the human tissue. However, they are not easy to obtain using conventional production methods. The present disclosure uses synthetic biology and structural biology technology to develop a macromolecular functional protein that has the triple helix structure of the human type II collagen and can perform the function of the human collagen. The inventors conducted a large-scale screening of functional regions. The first was the sequence screening. The Gly-X-Y repetitive gene sequence in the helical region of the natural type II collagen contains a large number of charged amino acids. These charges bind to the cell through interactions, so the regions that do not contain these important charge motifs are excluded. The second was to use computer-assisted protein structure prediction methods to help screen out the potential helical functional regions that have the most interchain hydrogen bond structures and can best stabilize trimer aggregation. The third was to use the prediction method based on the protein expression properties to screen out the functional regions of the human type II collagen with the highest protein expression, easy purification, and good stability. The fourth was to optimize the amino acid fragments of these regions by directly connecting them through n repetitions (the repetitions are to ensure that the molecular weight of the recombinant type II humanized collagen is within a certain range for easy purification and stability).

The present disclosure includes the core functional region screening, synthesis process, and application scenarios of the recombinant type II humanized collagen. The present disclosure for the first time develops a functional region screening and protein synthesis process for the recombinant type II humanized collagen that can be directly injected into the human body and can be used for cartilage repair.

The present disclosure is based in part on the following unexpected findings by the inventors. Compared with other recombinant type II humanized collagen, the recombinant type II humanized collagen HC2B-A10 has a higher expression level when recombinantly expressed, is easier to separate and purify, and has excellent cell adhesion effect compared with the bovine type I collagen.

In one aspect, the present disclosure provides a collagen or polypeptide comprising a plurality of repeating units comprising the amino acid sequence as shown in SEQ ID NO: 15 (gtpglpgvkghrgypgldgakgtpglpgvkghrgypgldgak) or the variant thereof having 80%, 85%, 90%, 91%, 92%, 93%, 94%, 95%, 96%, 97%, 98%, or 99% identity thereto, wherein each repeating unit is directly connected or separated by one or more amino acid residues. In one embodiment, the repeating unit may be an amino acid sequence obtained by mutation (substitution, insertion, deletion or addition) of one or more amino acid residues in the amino acid sequence of SEQ ID NO: 15.

In one embodiment, the number of the repeating unit is 1-20. In one embodiment, the number of the repeating unit is 6-10. In one embodiment, the number of the repeating unit is 2, 3, 4, 5, 6, 7, 8, 9, 10, 11, 12, 13, 14, 15, 16, 17, 18, 19 or 20.

In one embodiment, the collagen or polypeptide comprises the amino acid sequence as shown in SEQ ID NO: 10. In one embodiment, the collagen or polypeptide comprises the amino acid sequence as shown in SEQ ID NO: 10 or the amino acid sequence obtained by mutation (substitution, insertion, deletion or addition) of one or more amino acid residues of the amino acid sequence of SEQ ID NO: 3 or 10, or the variant having 80%, 85%, 90%, 91%, 92%, 93%, 94%, 95%, 96%, 97%, 98%, or 99% identity with the amino acid sequence as shown in SEQ ID NO: 10. In one embodiment, when the collagen or polypeptide sequence is mutated, the resulting collagen or polypeptide retains the functions of the present disclosure, such as cell adhesion, cartilage repair ability, etc.

In one aspect, the disclosure provides a polynucleotide encoding the collagen or polypeptide described herein.

In one embodiment, the polynucleotide comprises the nucleotide sequence set forth in SEQ ID NO: 29.

In one aspect, the disclosure provides a nucleic acid comprising the polynucleotide described herein. Optionally, the nucleic acid also comprises the nucleotides encoding a purification tag, such as His tag, GST tag, MBP tag, SUMO tag, or NusA tag. Optionally, the nucleic acid further comprises nucleotides encoding a leader sequence.

In one aspect, the disclosure provides a vector comprising the polynucleotide described herein or the nucleic acid described herein.

In one embodiment, the vector is an expression vector. In one embodiment, the vector comprises an expression control element, such as a promoter, terminator, and/or enhancer, operably linked to the polynucleotide or nucleic acid.

In one aspect, the disclosure provides a host cell comprising the polynucleotide described herein, the nucleic acid described herein, or the vector described herein. In one embodiment, the host cell is a bacterial, fungal, or animal cell. In one embodiment, the bacterium is E. coli. In one embodiment, the fungus is yeast, such as Saccharomyces cerevisiae.

In one aspect, the disclosure provides a method of producing the collagen or polypeptide described herein, comprising:
(1) Cultivate the host cell described herein under appropriate culture conditions;
(2) Harvest the host cell and/or culture medium comprising the collagen or peptides; and
(3) Purify the collagen or peptides.

In one aspect, the disclosure provides a composition comprising the collagen or polypeptide described herein. In one embodiment, the composition is a kit. In one embodiment, the composition is one or more of a biological dressing, a human bionic material, a plastic surgery material, an organoid culture material, a cardiovascular stent material, a coating material, a tissue injection filling material, an ophthalmic material, an obstetrics and gynecology biomaterial, a nerve repair regenerative material, a liver tissue material and a blood vessel repair regeneration material, a 3D printed artificial organ biomaterial, a cosmetic raw material, a pharmaceutical excipient, and a food additive. In one embodiment, the composition is an injectable or oral composition.

In one embodiment, the composition is a composition for cartilage repair. In one embodiment, the composition is an injectable composition for cartilage repair.

In one aspect, the disclosure provides a method for increasing cell adhesion, comprising contacting the cell with the collagen or polypeptide described herein, the polynucleotide described herein, the nucleic acid described herein, the vector described herein, the host cell described herein, and/or the composition described herein.

In one aspect, the disclosure provides use of the collagen or polypeptide described herein, the polynucleotide described herein, the nucleic acid described herein, the vector described herein, the host cell described herein, and/or the composition described herein in the manufacture of the kits, which are used for increasing cell adhesion or cartilage repair. In one aspect, the disclosure provides use of the collagen or polypeptide described herein, the polynucleotide described herein, the nucleic acid described herein, the vector described herein, the host cell described herein, and/or the composition described herein in one or more of a biological dressing, a human bionic material, a plastic surgery material, an organoid culture material, a cardiovascular stent material, a coating material, a tissue injection filling material, an ophthalmic material, an obstetrics and gynecology biomaterial, a nerve repair regenerative material, a liver tissue material, and a blood vessel repair regeneration material, a 3D printed artificial organ biomaterial, a cosmetic raw material, a pharmaceutical excipient, and a food additive.

Advantages of the disclosure include:

1. The present disclosure provides the core functional region and amino acid sequence of the recombinant type II humanized collagen.

2. The present disclosure successfully synthesizes the recombinant type II humanized collagen that can be injected into the human body for the first time for cartilage repair.

3. The amino acid composition of the recombinant type II humanized collagen produced is 100% identical to the corresponding part of the amino acid sequence of natural collagen, and will not cause immune rejection or allergic reactions when applied to the human body.

4. The preparation method of this disclosure is simple and can produce high-yield recombinant type II humanized collagen on a large scale.

5. In terms of expression, isolation, and purification effects, the recombinant type II humanized collagen HC2B-A10 achieves the best results compared with other recombinant type II humanized collagen.

6. The recombinant type II humanized collagen of the disclosure has better bioadhesion activity, that is, the recombinant type II humanized collagen HC2B-A10>bovine type I collagen.

### DESCRIPTION OF THE DRAWINGS

FIG. 1: Crude purification of the recombinant type II humanized collagens HC2B-a6 and HC2B-b5;

FIG. 2: Crude purification of the recombinant type II humanized collagens HC2B-b1, HC2B-a3, and HC2B-b3;

FIG. 3: Purification of the recombinant type II humanized collagens B3Q, HC2B-A8 and HC2B-A7;

FIG. 4: Purification of the recombinant type II humanized collagen HC2B-A10 and crude purification of the HC2B-B9;

FIG. 5: Purification of the recombinant type II humanized collagen HC2B-A9;

FIG. 6: Crude purification of the recombinant type II humanized collagens HC2B-B8, HC2B-B7, and HC2B-B10;

FIG. 7: Fine purification of the recombinant type II humanized collagens HC2B-B7 and HC2B-B9;

FIG. 8: Activity detection of the recombinant type II humanized collagen HC2B-A10;

FIG. 9: Map of pET-28a-Trx-His expression vector.

### DETAILED DESCRIPTION OF THE INVENTION

In order to make the purpose, technical solutions, and advantages of the present disclosure clearer, the technical solutions in the examples of the present disclosure will be clearly and completely described below in combination with the examples of the present disclosure. Obviously, the described examples are partial examples of the present disclosure, rather than all of the examples. Based on the examples of the present disclosure, all other examples obtained by ordinary technicians in this field without making any creative work fall within the scope of protection of the present disclosure.

Recombinant collagen is a new type of biological material with the same or similar amino acid sequence as human collagen, which is screened and prepared by using cutting-edge structural biology, genetic engineering, and other technologies and using the genetic coding of the functional region of a specific type of human collagen as a template.

As used herein, “polypeptide” refers to a plurality of amino acid residues linked by peptide bonds. In this disclosure, the collagen, recombinant collagen, recombinant type II humanized collagen, or polypeptide can be used interchangeably.

In this disclosure, the collagen or polypeptide may comprise one or more repeating units comprising the amino acid sequence as shown in SEQ ID NO: 15 or the amino acid sequence after mutation (substitution, addition, insertion, or deletion) of one or more amino acid residues in the amino acid sequence. The number of the repeating unit may be 1-20. For example, the number of the repeating unit is 2, 3, 4, 5, 6, 7, 8, 9, 10, 11, 12, 13, 14, 15, 16, 17, 18, 19 or 20. In particular, the mutations may be substitutions, such as conservative amino acid substitutions. The amino acid sequence of SEQ ID NO: 15 is gtpglpgvkghrgypgldgakgtpglpgvkghrgypgldgak. Each repeating unit may be directly linked or separated by one or more amino acid residues.

In the case where the collagen or polypeptide sequence is mutated or there is a spacer sequence, the resulting collagen or polypeptide retains the functions of the present disclosure, such as cell adhesion, cartilage repair ability, etc.

The collagen or polypeptide of the disclosure may be synthetic or may be recombinantly expressed. In the case of recombinant expression, the collagen or polypeptide of the disclosure may be encoded by the polynucleotide. The polynucleotide can be codon-optimized for the host cell in which expression is performed. The polynucleotide encoding the collagen or polypeptide can be operably linked to the expression control element, such as the promoter, terminator, and/or enhancer to constitute nucleic acid or expression cassettes. The nucleic acid may also comprise nucleotides encoding a purification tag, such as a His tag, a GST tag, an MBP tag, a SUMO tag, or a NusA tag, or nucleotides encoding a leader sequence to facilitate purification or secretion of the collagen or polypeptide.

As used herein, the term “vector” is a nucleic acid vehicle into which a polynucleotide can be inserted. When a vector can express the protein encoded by the inserted polynucleotide, the vector is called an expression vector. The vector can be introduced into the host cell by transformation, transduction, or transfection, so that the genetic material elements it carries are expressed in the host cell. The vector is well known to those skilled in the art, and includes but is not limited to plasmids; phagemids; cosmids; artificial chromosomes, such as yeast artificial chromosomes (YACs), bacterial artificial chromosomes (BACs) or P1-derived artificial chromosomes (PACs); bacteriophages such as λ phage or M13 phage and animal viruses, etc. The vector may contain a variety of elements for controlling expression, including but not limited to promoter sequences, transcription initiation sequences, enhancer sequences, selection elements, and reporter genes. In addition, the vector may also contain an origin of replication site. The vector may comprise the nucleic acid of the disclosure for easy introduction into the cell for expression. The vector may comprise the expression control element, such as the promoter, terminator and/or enhancer, operably linked to the said nucleic acid.

As used herein, the term “host cell” is a cell into which the nucleic acid molecules have been introduced by molecular biology techniques. These techniques include transfection with a viral vector, transformation with a plasmid vector, and accelerated introduction of naked DNA by electroporation, lipofection, and particle gun. The host cell can be a eukaryotic cell or a prokaryotic cell. The eukaryotic cell is, for example, a yeast cell, an animal cell and/or an insect cell. The prokaryotic cell can be an E. coli cell.

The present disclosure also provides a method of producing the collagen or polypeptide, comprising: (1) culturing the host cell herein under suitable culture conditions; (2) harvesting the host cell and/or culture medium comprising the collagen or polypeptide; and (3) purify the collagen or polypeptide. The method of the present disclosure may include the step of enzymatically cleaving the tag.

The collagen or polypeptide of the present disclosure can be prepared into a composition or kit. The composition or kit may be a composition or kit for tissue filling and/or expansion. The composition or kit may also comprise auxiliary substances. The composition of the present disclosure may be a cartilage repair agent comprising the collagen or polypeptide described herein. The composition of the disclosure may be injectable. The composition of the present disclosure can be a structural material of the human body, for example, can be used for cartilage repair, and does not cause an immune response in the human body.

As used herein, the degree of relatedness between two amino acid sequences or between two nucleotide sequences is described by the parameter “sequence identity.” For the purposes of this disclosure, the sequence identity between two amino acids is determined by Needleman-Wunsch Algorithm implemented by the Needle program (Needleman and Wunsch, 1970, J. Mol. Biol. 48:443-453) of the EMBOSS software package (EMBOSS: The European Molecular Biology Open Software Suite, Rice et al, 2000, Trends Genet. 16:276-277) (Version 5.0.0 or later preferred). The parameters used are the gap opening penalty of 10, the gap extension penalty of 0.5, and the EBLOSUM62 (EMBOSS version of BLOSUM62) substitution matrix. Needle's output labeled “Longest Identity” (obtained using the non-simplified option) was used as the percent identity and calculated as follows:

(identical residues×100)/(alignment length−total number of gaps in the alignment)

For the purposes of this disclosure, the sequence identity between two deoxynucleotide sequences is determined by the Needleman-Wunsch Algorithm implemented by the Needle program (Needleman and Wunsch, 1970, J. Mol. Biol. 48:443-453) of the EMBOSS software package (EMBOSS: The European Molecular Biology Open Software Suite, Rice et al, 2000, Trends Genet. 16:276-277) (Version 5.0.0 or later preferred). The parameters used are the gap opening penalty of 10, the gap extension penalty of 0.5, and the EDNAFULL (EMBOSS version of NCBI NUC4.4) substitution matrix. Needle's output labeled “Longest Identity” (obtained using the non-simplified option) was used as the percent identity and calculated as follows:

(identical deoxyribonucleotides×100)/(alignment length−total number of gaps in the alignment)

Herein, the repeating unit of the collagen or polypeptide of the present disclosure or the collagen or polypeptide may contain certain mutations. For example, one or more of the amino acid sequences of these parts may contain a substitution, deletion, addition or insertion of amino acid residues. In the context of amino acid mutations, the “plurality” can be 2-40, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 31, 32, 33, 34, 35, 36, 37, 38 or 39, or any range of values between them.

That is, the present disclosure can use repeat unit variants as long as the variant retains the activity of promoting cell adhesion. In particular, the variants may have a certain percentage identity with a specified sequence (any of the collagen or polypeptide sequences described herein), such as 80%, 85%, 90%, 91%, 92%, 93%, 94%, 95%, 96%, 97%, 98%, 99% identity. The designated sequence may be any sequence of the disclosure, such as SEQ ID Nos: 1-16, but it is preferred that these variants retain the intended function, such as increasing cell adhesion or cartilage repair.

### Methods of the Present Disclosure

In view of the current research status, the present disclosure provides a method for biosynthesizing recombinant type II humanized collagen, that is, a method for preparing human body structural materials. The method may include one or more of the following steps: (1) screening of functional regions and construction of bacterial strains; (2) large-scale biological fermentation culture and induced expression of proteins; (3) purification of the humanized type II collagen and optional enzymatic cleavage.

Screening of functional regions and construction of bacterial strains can be carried out as follows: (1) performing large-scale functional region screening to obtain the functional region of the target gene; (2) inserting the obtained functional region of the target gene into the PET-28a-Trx-His expression vector to obtain a recombinant expression plasmid; (3) transferring the recombinant expression plasmid into the E. coli competent cell BL21 (DE3) to screen and obtain positive E. coli genetically engineered bacteria.

Large-scale biological fermentation can be carried out as follows: the positive E. coli genetically engineered bacteria obtained by screening are added to a shake flask containing an antibiotic stock solution and cultured in a constant temperature shaker at 220 rpm and 37° C.

The induced expression of the protein can be carried out as follows: (1) cooling the shake flask after culture to 16-30° C.; (2) adding IPTG mother solution to induce expression; (3) placing the bacterial solution after induction of expression in a centrifuge bottle, centrifuging at 6000 rpm and 4° C. for 12 min before collecting the bacteria.

The purification of the humanized type II collagen and optional enzymatic cleavage can be carried out as follows: (1) crudely purifying the humanized type II collagen on a Ni affinity chromatography column; (2) adding TEV enzyme for enzymatic cleavage at a certain ratio; (3) finely purifying the humanized type II collagen on an ion exchange column.

The screened functional regions are as follows:

(1) The amino acid sequence of HC2B-a3:
(SEQ ID NO: 1)
gkpgddgeagkpgkagergppgpqgargfpgtpglpgvkghrgypgldgakgeag
apgvkgesgspgengspgpmgprglpgergrtgpagaagargndgqp;
(2) The amino acid sequence of HC2B-a6:
(SEQ ID NO: 2)
gkpgkagergppgpqgargfpgtpglpgvkghrgkpgkagergppgpqgargfpg
tpglpgvkghrgkpgkagergppgpqgargfpgtpglpgvkghrgkpgkagergp
pgpqgargfpgtpglpgvkghrgkpgkagergppgpqgargfpgtpglpgvkghr
gkpgkagergppgpqgargfpgtpglpgvkghrgkpgkagerg;
(3) The amino acid sequence of HC2B-b1:
(SEQ ID NO: 3)
gepgregspgadgppgrdgaagvkgdrgetgavgapgapgppgspgpagptgkqg
drgeagaqgpmgpsgpagargiqgpqgprgdkgeagepgerglkghrgftglqgl
pgppgpsgdqgasgpagpsgprgppgpvgpsgkdgangipgpigppgprgrsget
gpa;
(4) The amino acid sequence of HC2B-b3:
(SEQ ID NO: 4)
gspgpagptgkqgdrgeagaqgpmgpsgpagargiqgpqgprgdkgeagepgerg
lkghrgftglqglpgppgpsgdqgasgpagpsgpr;
(5) The amino acid sequence of HC2B-b5:
(SEQ ID NO: 5)
gargiqgpqgprgdkgeagepgerglkghrgftglqglpgppgpsgargiqgpqg
prgdkgeagepgerglkghrgftglqglpgppgpsgargiqgpqgprgdkgeage
pgerglkghrgftglqglpgppgpsgargiqgpqgprgdkgeagepgerglkghr
gftglqglpgppgpsgargiqgpqgprgdkgeagepgerglkghrgftglqglpg
ppgpsgargiqgpqgprgdkgeagepgerglkghrgftglqglpgppgpsgargi
qgpqgprgdkgeagepgerglkghrgftglqglpgppgpsgargiqgpqgprgdk
geagepgerglkghrgftglqglpgppgps;
(6) The amino acid sequence of B3Q:
(SEQ ID NO: 6)
gspgpagptgkqgdrgeagaqgpmgpsgpagargiqgpqgprgdkgeagepgerg
lkghrgftglqglpgppgpsgdqgasgpagpsgprgspgpagptgkqgdrgeaga
qgpmgpsgpagargiqgpqgprgdkgeagepgerglkghrgftglqglpgppgps
gdqgasgpagpsgprgspgpagptgkqgdrgeagaqgpmgpsgpagargiqgpqg
prgdkgeagepgerglkghrgftglqglpgppgpsgdqgasgpagpsgprgspgp
agptgkqgdrgeagaqgpmgpsgpagargiqgpqgprgdkgeagepgerglkghr
gftglqglpgppgpsgdqgasgpagpsgpr;
(7) The amino acid sequence of HC2B-A7:
(SEQ ID NO: 7)
gpqgargfpgtpglpgvkghrgypgldgakgpqgargfpgtpglpgvkghrgypg
ldgakgpqgargfpgtpglpgvkghrgypgldgakgpqgargfpgtpglpgvkgh
rgypgldgakgpqgargfpgtpglpgvkghrgypgldgakgpqgargfpgtpglp
gvkghrgypgldgakgpqgargfpgtpglpgvkghrgypgldgakgpqgargfpg
tpglpgvkghrgypgldgakgpqgargfpgtpglpgvkghrgypgldgakgpqga
rgfpgtpglpgvkghrgypgldgak;
(8) The amino acid sequence of HC2B-A8:
(SEQ ID NO: 8)
gargfpgtpglpgvkghrgypgldgakgargfpgtpglpgvkghrgypgldgakg
argfpgtpglpgvkghrgypgldgakgargfpgtpglpgvkghrgypgldgakga
rgfpgtpglpgvkghrgypgldgakgargfpgtpglpgvkghrgypgldgakgar
gfpgtpglpgvkghrgypgldgakgargfpgtpglpgvkghrgypgldgakgarg
fpgtpglpgvkghrgypgldgakgargfpgtpglpgvkghrgypgldgakgargf
pgtpglpgvkghrgypgldgakgargfpgtpglpgvkghrgypgldgak;
(9) The amino acid sequence of HC2B-A9:
(SEQ ID NO: 9)
gfgfpgtpglpgvkghrgypgldgakgfpgtpglpgvkghrgypgldgakgfpgt
pglpgvkghrgypgldgakgfpgtpglpgvkghrgypgldgakgfpgtpglpgvk
ghrgypgldgakgfpgtpglpgvkghrgypgldgakgfpgtpglpgvkghrgypg
ldgakgfpgtpglpgvkghrgypgldgakgfpgtpglpgvkghrgypgldgakgf
pgtpglpgvkghrgypgldgakgfpgtpglpgvkghrgypgldgakgfpgtpglp
gvkghrgypgldgakgfpgtpglpgvkghrgypgldgakgfpgtpglpgvkghrg
ypgldgak;
(10) The amino acid sequence of HC2B-A10:
(SEQ ID NO: 10)
gtpglpgvkghrgypgldgakgtpglpgvkghrgypgldgakgtpglpgvkghrg
ypgldgakgtpglpgvkghrgypgldgakgtpglpgvkghrgypgldgakgtpgl
pgvkghrgypgldgakgtpglpgvkghrgypgldgakgtpglpgvkghrgypgld
gakgtpglpgvkghrgypgldgakgtpglpgvkghrgypgldgakgtpglpgvkg
hrgypgldgakgtpglpgvkghrgypgldgakgtpglpgvkghrgypgldgakgt
pglpgvkghrgypgldgakgtpglpgvkghrgypgldgakgtpglpgvkghrgyp
gldgak;
(11) The amino acid sequence of HC2B-B7:
(SEQ ID NO: 11)
gerglkghrgftglqglpgppgpsgdqgasgpagpsgprgerglkghrgftglqg
lpgppgpsgdqgasgpagpsgprgerglkghrgftglqglpgppgpsgdqgasgp
agpsgprgerglkghrgftglqglpgppgpsgdqgasgpagpsgprgerglkghr
gftglqglpgppgpsgdqgasgpagpsgprgerglkghrgftglqglpgppgpsg
dqgasgpagpsgprgerglkghrgftglqglpgppgpsgdqgasgpagpsgprge
rglkghrgftglqglpgppgpsgdqgasgpagpsgpr;
(12) The amino acid sequence of HC2B-B8
(SEQ ID NO: 12)
gerglkghrgftglqglpgppgpsgdqgasgpagerglkghrgftglqglpgppg
psgdqgasgpagerglkghrgftglqglpgppgpsgdqgasgpagerglkghrgf
tglqglpgppgpsgdqgasgpagerglkghrgftglqglpgppgpsgdqgasgpa
gerglkghrgftglqglpgppgpsgdqgasgpagerglkghrgftglqglpgppg
psgdqgasgpagerglkghrgftglqglpgppgpsgdqgasgpagerglkghrgf
tglqglpgppgpsgdqgasgpagerglkghrgftglqglpgppgpsgdqgasgpa;
(13) The amino acid sequence of HC2B-B9:
(SEQ ID NO: 13)
gerglkghrgftglqglpgppgpsgdqgerglkghrgftglqglpgppgpsgdqg
erglkghrgftglqglpgppgpsgdqgerglkghrgftglqglpgppgpsgdqge
rglkghrgftglqglpgppgpsgdqgerglkghrgftglqglpgppgpsgdqger
glkghrgftglqglpgppgpsgdqgerglkghrgftglqglpgppgpsgdqgerg
lkghrgftglqglpgppgpsgdqgerglkghrgftglqglpgppgpsgdqgergl
kghrgftglqglpgppgpsgdqgerglkghrgftglqglpgppgpsgdq;
(14) The amino acid sequence of HC2B-B10:
(SEQ ID NO: 14)
gerglkghrgftglqglpgppgerglkghrgftglqglpgppgerglkghrgftg
lqglpgppgerglkghrgftglqglpgppgerglkghrgftglqglpgppgergl
kghrgftglqglpgppgerglkghrgftglqglpgppgerglkghrgftglqglp
gppgerglkghrgftglqglpgppgerglkghrgftglqglpgppgerglkghrg
ftglqglpgppgerglkghrgftglqglpgppgerglkghrgftglqglpgppge
rglkghrgftglqglpgppgerglkghrgftglqglpgppgerglkghrgftglq
glpgpp.

The amino acid sequence of the recombinant type II humanized collagen prepared by the present disclosure can be derived from the functional region of human natural type II collagen, including this functional region and similar functional regions, the proteins with mutated and modified amino acid sequences.

The recombinant type II humanized collagen prepared by the disclosure completely matches the amino acid sequence of human collagen, so it can be directly injected into the human body for cartilage repair without producing the immunogenic response.

### The Collagen or Polypeptide of the Disclosure

The present disclosure provides the collagen or polypeptide, which comprises the amino acid sequence of any one of SEQ ID NO: 1-14 or the amino acid sequence of which the amino acid has been mutated. Mutations can be substitution, addition, deletion, or insertion. Preferably, the substitution may be a retained substitution. The collagen or polypeptide of the disclosure can be derived from the peptide fragments derived from the type II human collagen. The collagen or polypeptide of the disclosure may comprise a plurality of peptide fragments obtained in this way. The peptide segments can be connected through a linker or directly to form the collagen or polypeptide of the present disclosure. The linker can be one or more amino acid residues. For example, the linker may be a flexible linker commonly used in the art.

The present disclosure provides multiple repeating units, as shown in the underlined amino acid sequences of the examples. In particular, the present disclosure provides the repeating unit of the amino acid sequence of SEQ ID NO: 15. The collagen or polypeptide of the present disclosure may contain multiple repeating units, such as 2-30, 2, 3, 4, 5, 6, 7, 8, 9, 10, 11, 12, 13, 14, 15, 16, 17, 18, 19, 20, 21, 22, 23, 24, 25, 26, 27, 28 or 29.

### EXAMPLES

The following examples are provided to illustrate the invention. Those skilled in the art should understand that the examples are only illustrative but not restrictive. The invention is limited only by the scope of the appended claims.

### Example 1: Construction and Expression of the Recombinant Type II Humanized Collagen

1. Performing large-scale functional region screening. The first was the sequence screening. The Gly-X-Y repetitive gene sequence in the helical region of the natural type II collagen contains a large number of charged amino acids. These charges bind to the cell through interactions, so the regions that do not contain these important charge motifs are excluded. The second was to use computer-assisted protein structure prediction methods to help screen out the potential helical functional regions that have the most interchain hydrogen bond structures and can best stabilize trimer aggregation. The third was to use the prediction method based on the protein expression properties to screen out the functional regions of the human type II collagen with the highest protein expression, easy purification, and good stability. The fourth was to optimize the amino acid fragments of these regions by directly connecting them through n repetitions (the repetitions are to ensure that the molecular weight of the recombinant type II humanized collagen is within a certain range for easy purification and stability); the following target gene functional regions of different recombinant type II humanized collagens were obtained:

(1)
the amino acid sequence of HC2B-a3:
(SEQ ID NO: 1)
gkpgddgeagkpgkagergppgpqgargfpgtpglpgvkghrgypgldgakgeag
apgvkgesgspgengspgpmgprglpgergrtgpagaagargndgqp;
(2)
the amino acid sequence of HC2B-a6:
(SEQ ID NO: 2, the underlined is the repeating unit portion)
gkpgkagergppgpqgargfpgtpglpgvkghrgkpgkagergppgpqgargfpg
tpglpgvkghrgkpgkagergppgpqgargfpgtpglpgvkghrgkpgkagergp
pgpqgargfpgtpglpgvkghrgkpgkagergppgpqgargfpgtpglpgvkghr
gkpgkagergppgpqgargfpgtpglpgvkghrgkpgkagerg;
(3)
the amino acid sequence of HC2B-b1:
(SEQ ID NO: 3)
gepgregspgadgppgrdgaagvkgdrgetgavgapgapgppgspgpagptgkqg
drgeagaqgpmgpsgpagargiqgpqgprgdkgeagepgerglkghrgftglqgl
pgppgpsgdqgasgpagpsgprgppgpvgpsgkdgangipgpigppgprgrsget
gpa;
(4)
the amino acid sequence of HC2B-b3:
(SEQ ID NO: 4)
gspgpagptgkqgdrgeagaqgpmgpsgpagargiqgpqgprgdkgeagepgerg
lkghrgftglqglpgppgpsgdqgasgpagpsgpr;
(5)
the amino acid sequence of HC2B-b5:
(SEQ ID NO: 5, the underlined is the repeating unit portion)
gargiqgpqgprgdkgeagepgerglkghrgftglqglpgppgpsgargiqgpqg
prgdkgeagepgerglkghrgftglqglpgppgpsgargiqgpqgprgdkgeage
pgerglkghrgftglqglpgppgpsgargiqgpqgprgdkgeagepgerglkghr
gftglqglpgppgpsgargiqgpqgprgdkgeagepgerglkghrgftglqglpg
ppgpsgargiqgpqgprgdkgeagepgerglkghrgftglqglpgppgpsgargi
qgpqgprgdkgeagepgerglkghrgftglqglpgppgpsgargiqgpqgprgdk
geagepgerglkghrgftglqglpgppgps;
(6)
the amino acid sequence of B3Q:
(SEQ ID NO: 6, the underlined is the repeating unit portion)
gspgpagptgkqgdrgeagaqgpmgpsgpagargiqgpqgprgdkgeagepgerG
lkghrgftglqglpgppgpsgdqgasgpagpsgprgspgpagptgkqgdrgeaga
qgpmgpsgpagargiqgpqgprgdkgeagepgerglkghrgftglqglpgppgps
gdqgasgpagpsgprgspgpagptgkqgdrgeagaqgpmgpsgpagargiqgpqg
prgdkgeagepgerglkghrgftglqglpgppgpsgdqgasgpagpsgprgspgp
agptgkqgdrgeagaqgpmgpsgpagargiqgpqgprgdkgeagepgerglkghr
gftglqglpgppgpsgdqgasgpagpsgpr;
(7)
HC2B-A7:
(SEQ ID NO: 7, the underlined is the repeating unit portion)
gpqgargfpgtpglpgvkghrgypgldgakgpqgargfpgtpglpgvkghrgypg
ldgakgpqgargfpgtpglpgvkghrgypgldgakgpqgargfpgtpglpgvkgh
rgypgldgakgpqgargfpgtpglpgvkghrgypgldgakgpqgargfpgtpglp
gvkghrgypgldgakgpqgargfpgtpglpgvkghrgypgldgakgpqgargfpg
tpglpgvkghrgypgldgakgpqgargfpgtpglpgvkghrgypgldgakgpqga
rgfpgtpglpgvkghrgypgldgak;
(8)
HC2B-A8:
(SEQ ID NO: 8, the underlined is the repeating unit portion)
gargfpgtpglpgvkghrgypgldgakgargfpgtpglpgvkghrgypgldgakg
argfpgtpglpgvkghrgypgldgakgargfpgtpglpgvkghrgypgldgakga
rgfpgtpglpgvkghrgypgldgakgargfpgtpglpgvkghrgypgldgakgar
gfpgtpglpgvkghrgypgldgakgargfpgtpglpgvkghrgypgldgakgarg
fpgtpglpgvkghrgypgldgakgargfpgtpglpgvkghrgypgldgakgargf
pgtpglpgvkghrgypgldgakgargfpgtpglpgvkghrgypgldgak;
(9)
HC2B-A9:
(SEQ ID NO: 9, the underlined is the repeating unit portion)
gfpgtpglpgvkghrgypgldgakgfpgtpglpgVkghrgypgldgakgfpgtpg
lpgvkghrgypgldgakgfpgtpglpgvkghrgypgldgakgfpgtpglpgvkgh
rgypgldgakgfpgtpglpgvkghrgypgldgakgfpgtpglpgvkghrgypgld
gakgfpgtpglpgvkghrgypgldgakgfpgtpglpgvkghrgypgldgakgfpg
tpglpgvkghrgypgldgakgfpgtpglpgvkghrgypgldgakgfpgtpglpgv
kghrgypgldgakgfpgtpglpgvkghrgypgldgakgfpgtpglpgvkghrgyp
gldgak;
(10) the amino acid sequence of HC2B-A10:
(SEQ ID NO: 10)
gtpglpgvkghrgypgldgakgtpglpgvkghrgypgldgak
Gtpglpgvkghrgypgldgakgtpglpgvkghrgypgldgak
Gtpglpgvkghrgypgldgakgtpglpgvkghrgypgldgak
Gtpglpgvkghrgypgldgakgtpglpgvkghrgypgldgak
Gtpglpgvkghrgypgldgakgtpglpgvkghrgypgldgak
Gtpglpgvkghrgypgldgakgtpglpgvkghrgypgldgak
Gtpglpgvkghrgypgldgakgtpglpgvkghrgypgldgak
Gtpglpgvkghrgypgldgakgtpglpgvkghrgypgldgak;
(11) the amino acid sequence of HC2B-B7:
(SEQ ID NO: 11, the underlined is the repeating unit portion)
gerglkghrgftglqglpgppgpsgdqgasgpagpsgprgerglkghrgftglqg
lpgppgpsgdqgasgpagpsgprgerglkghrgftglqglpgppgpsgdqgasgp
agpsgprgerglkghrgftglqglpgppgpsgdqgasgpagpsgprgerglkghr
gftglqglpgppgpsgdqgasgpagpsgprgerglkghrgftglqglpgppgpsg
dqgasgpagpsgprgerglkghrgftglqglpgppgpsgdqgasgpagpsgprge
rglkghrgftglqglpgppgpsgdqgasgpagpsgpr;
(12)
the amino acid sequence of HC2B-B8:
(SEQ ID NO: 12)
gasgpagerglkghrgftglqglpgppgpsgdqgasgpagerglkghrgftglqg
lpgppgpsgdqgasgpagerglkghrgftglqglpgppgpsgdqgasgpagergl
kghrgftglqglpgppgpsgdqgasgpagerglkghrgftglqglpgppgpsgdq
gasgpagerglkghrgftglqglpgppgpsgdqgasgpagerglkghrgftglqg
lpgppgpsgdqgasgpagerglkghrgftglqglpgppgpsgdqgasgpagergl
kghrgftglqglpgppgpsgdqgasgpagerglkghrgftglqglpgppgpsgdq;
(13)
the amino acid sequence of HC2B-B9:
(SEQ ID NO: 13)
gerglkghrgftglqglpgppgpsgdqgerglkghrgftglqglpgppgpsgdqg
erglkghrgftglqglpgppgpsgdqgerglkghrgftglqglpgppgpsgdqge
rglkghrgftglqglpgppgpsgdqgerglkghrgftglqglpgppgpsgdqger
glkghrgftglqglpgppgpsgdqgerglkghrgftglqglpgppgpsgdqgerg
lkghrgftglqglpgppgpsgdqgerglkghrgftglqglpgppgpsgdqgergl
kghrgftglqglpgppgpsgdqgerglkghrgftglqglpgppgpsgdq;
(14)
the amino acid sequence of HC2B-B10:
(SEQ ID NO: 14)
gerglkghrgftglqglpgppgerglkghrgftglqglpgppgerglkghrgftg
lqglpgppgerglkghrgftglqglpgppgerglkghrgftglqglpgppgergl
kghrgftglqglpgppgerglkghrgftglqglpgppgerglkghrgftglqglp
gppgerglkghrgftglqglpgppgerglkghrgftglqglpgppgerglkghrg
ftglqglpgppgerglkghrgftglqglpgppgerglkghrgftglqglpgppge
rglkghrgftglqglpgppgerglkghrgftglqglpgppgerglkghrgftglq
glpgpp.

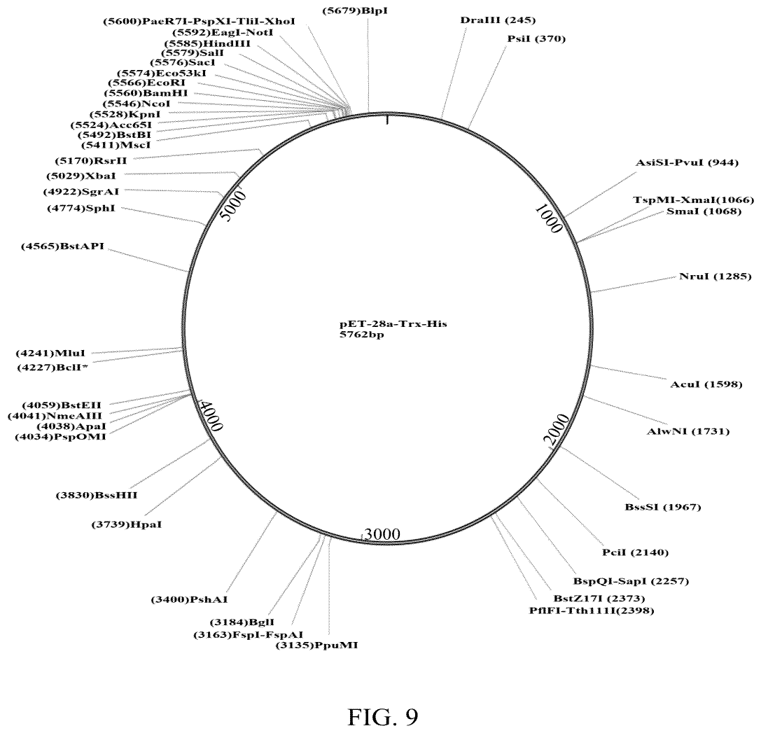

2. The synthesized gene functional region was inserted into the pET-28a-Trx-His expression vector (FIG. 9) to obtain the corresponding recombinant expression plasmid.

3. The successfully constructed expression plasmid was transformed into E. coli competent cell BL21 (DE3). The specific process was as follows: (1) The competent E. coli cells BL21 (DE3) were taken out from the ultra-low temperature refrigerator and placed on the ice. When the cells were half melted, 2 μl of the plasmid to be transformed was taken out and added to the E. coli competent cells BL21 (DE3), and mixed slightly 2-3 times. (2) The mixture was placed on the ice for 30 min, then heated shock in a 42° C. water bath for 45-90s, and placed on the ice bath for 2 min after removal. (3) The mixture was transferred to a biosafety cabinet, added with 700 μl of liquid LB medium, and then incubated at 37° C. and 220 rpm for 60 min. (4) 200 μl of the bacterial solution was taken and spread evenly on an LB plate containing kanamycin sulfate (50 mg/L). (5) The plate was incubated in a 37° C. incubator for 15-17 h until colonies of uniform size grew.

4. 5-6 single colonies were picked from the transformed LB plate and placed in a shake flask containing antibiotic stock solution (ampicillin, 100 mg/L), and cultured in a constant temperature shaker at 220 rpm and 37° C. for a certain period until appeared foggy. The cultured shake flask was cooled to 16-30° C., added with IPTG (0.5 mM) to induce expression for a period of time before the bacterial solution was dispensed into centrifuge bottles and centrifuged at 6000 rpm and 4° C. for 12 min. The bacterial cells were collected, the weight of the bacterial cells was recorded, and the samples were taken for electrophoresis detection.

5. The collected bacteria were resuspended in a balanced working solution (200 mM sodium chloride, 25 mM Tris, 20 mM imidazole), the bacterial solution was cooled to ≤15° C., homogenized twice or ultrasonically disrupted cells. The bacterial solution was collected after completion. The bacterial solution after cell disruption was dispensed into centrifuge bottles, centrifuged at 17000 rpm and 4° C. for 30 min, and the supernatant was collected.

6. The recombinant type II humanized collagen was purified and enzymatically cleaved. The specific process was as follows: (1) Crude purification: a. Column equilibration: the column was equilibrated with the equilibrium solution (200 mM sodium chloride, 25 mM Tris, 20 mM imidazole) at a flow rate of 10 mL/min. b. Loading: The supernatant after centrifugation was added to the column until the liquid flowed out at a flow rate of 5 mL/min. c. Washing the impure proteins: 100 mL of washing solution (200 mM sodium chloride, 25 mM Tris, 20 mM imidazole) was added until the liquid flowed out at a flow rate of 10 mL/min. d. Collection of the target protein: 20 mL of eluent (200 mM sodium chloride, 25 mM Tris, 250 mM imidazole) was added, with a flow rate of 10 mL/min, the flow-through fluid was collected, the protein concentration was detected by UV-Vis and calculated according to the following formula (C (mg/ml)=A280×dilution factor×extinction coefficient), and the electrophoresis detection was conducted. e. The column was cleaned with IM imidazole working solution at a flow rate of 10 mL/min. (2) Enzymically cleavage: the TEV enzyme was added according to the ratio of total protein amount to the total amount of TEV enzyme at 20:1, and enzymatically cleaved at 16° C. for 2 h. The digested protein solution was put into a dialysis bag, dialyzed at 4° C. for 2 h, and then transferred to new dialysate (20 mM sodium chloride, 20 mM Tris) for overnight dialysis at 4° C. (3) Fine purification: a. Column equilibration: the column was equilibrated with solution A (20 mM Tris, 20 mM sodium chloride) at a flow rate of 10 ml/min. b. Loading: The flow rate was 5 ml/min, the sample was loaded and the flow-through sample was collected, the electrophoresis detection was conducted, and the protein was stored in a 4° C. environment. c. Elution: the column was washed with solution B (1 M sodium chloride, 20 mM Tris) for 5 CV. d. Washing the column.

### Test Results:

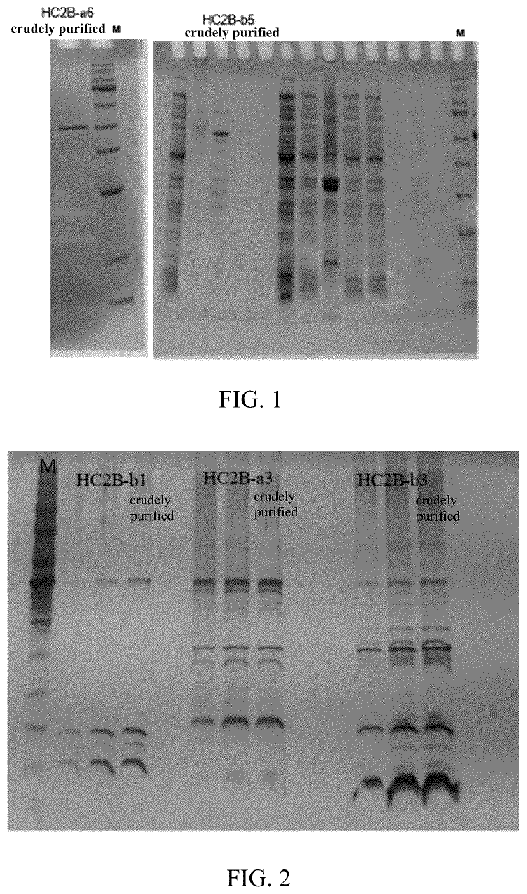

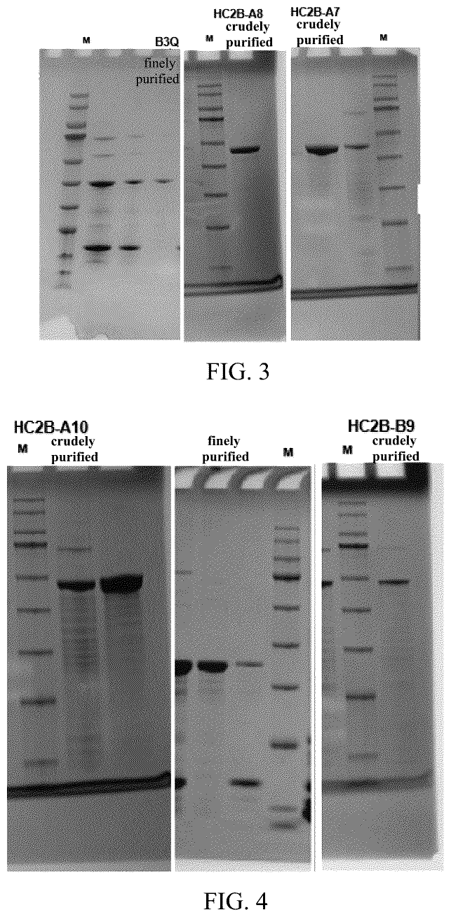

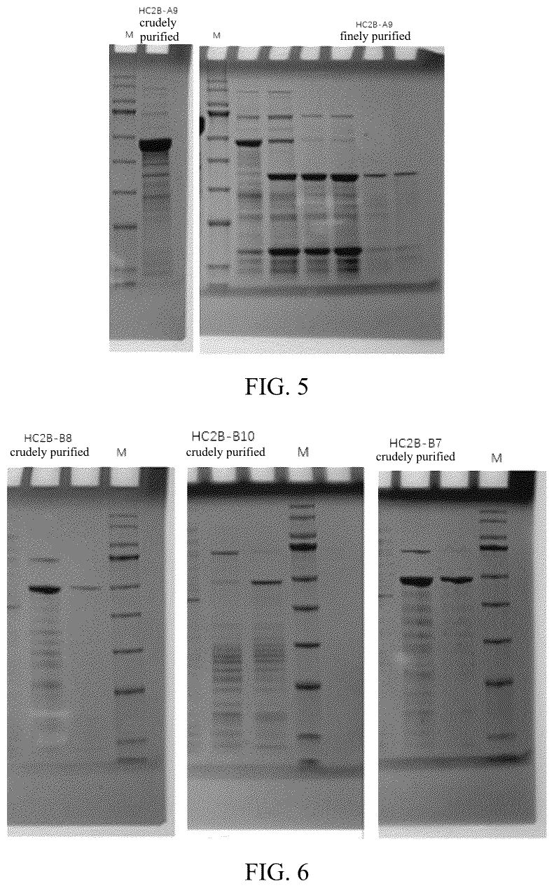

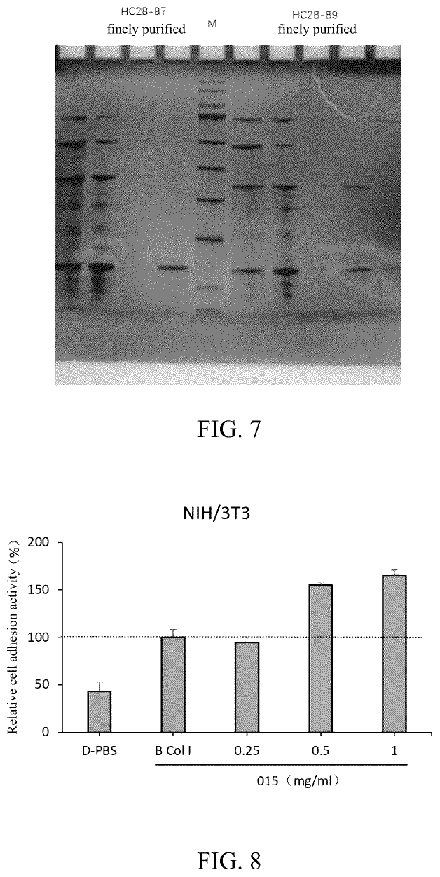

The electrophoresis detection results involved in the preparation process of each recombinant protein are shown in FIGS. 1 to 7. FIG. 1 shows the electrophoresis detection results of the recombinant type II humanized collagens HC2B-a6 and HC2B-b5, indicating that the expression amount of the target protein is low after crude purification. FIG. 2 shows the electrophoresis detection results of the recombinant type II humanized collagens HC2B-a3, HC2B-b1, and HC2B-b3, indicating that there is a lot of impure proteins after crude purification and fine purification of the protein. FIG. 3 shows the electrophoresis detection results of the recombinant type II humanized collagens HC2B-HC2B-A7 and HC2B-HC2B-A8, indicating that the expression amount of the target protein is low after crude purification. FIG. 3 also shows the electrophoresis detection results of the recombinant type II humanized collagen B3Q, indicating that the amount of the protein is low after fine purification. FIG. 4 shows the electrophoresis detection results of the recombinant type II humanized collagen HC2B-A10, indicating that the expression level of crudely purified proteins is relatively high, the enzymatic digestion effect is better, and the protein purity is high after fine purification (left and middle panel of FIG. 4). FIGS. 4 and 7 also show the electrophoresis detection results of the recombinant type II humanized collagen HC2B-B9, indicating that the expression level of crudely purified proteins is relatively low with more impurity bands, and the enzymatic digestion effect is poor. FIG. 5 shows the electrophoresis detection results of the recombinant type II humanized collagen HC2B-A9, indicating that the expression level of crudely purified proteins is relatively high with more impurity bands and lower purity. The left panel of FIG. 6 shows the electrophoresis detection results of the recombinant type II humanized collagen HC2B-B8, indicating that the expression level of crudely purified proteins is relatively low with many impurity bands, without subsequent fine purification. The right panel of FIG. 6 and FIG. 7 show the electrophoresis detection results of the recombinant type II humanized collagen HC2B-B7, indicating that the expression level of crudely purified proteins is relatively low with more impurity bands, and the enzymatic digestion effect is poor. The middle panel of FIG. 6 shows the electrophoresis detection of the recombinant type II humanized collagen HC2B-B10. The results indicate that the expression level of crudely purified proteins is relatively low with many impurity bands, without subsequent fine purification.

Therefore, in terms of expression, separation and purification effects, the recombinant type II humanized collagen HC2B-A10 achieved the best effect compared with other recombinant type II humanized collagen.

### Example 2: Detection of Biological Activity of Recombinant Type II Humanized Collagen HC2B-A10

The methods of collagen activity detection can refer to Juming Yao, Satoshi Yanagisawa, Tetsuo Asakura, Design, Expression and Characterization of Collagen-Like Proteins Based on the Cell Adhesive and Crosslinking Sequences Derived from Native Collagens, J Biochem. 136, 643-649 (2004). The specific implementation methods were as follows:

(1) the concentration of the protein samples to be tested was detected with ultraviolet absorption, including the bovine type I collagen standard (Sigma, Cat. No.: 380002), the recombinant type II humanized collagen HC2B-A10 with good purification and enzyme digestion effects provided by the present disclosure (No. 015).

Specifically, the ultraviolet absorption of the samples at 215 nm and 225 nm was measured respectively, and the protein concentrations were calculated using the empirical formula C (μg/mL)=144×(A215-A225). Note that the detection needed to be performed when A215<1.5. The principle of this method is that the characteristic absorption of peptide bonds is detected under far-ultraviolet light, which is not affected by the chromophore content, with few interfering substances and is easy to operate, so that is suitable for the detection of human collagen and analogs thereof that do not develop color in Coomassie brilliant blue. (See the reference Walker JM. The Protein Protocols Handbook, second edition. HumanaPress. 43-45). After the protein concentration was detected, the concentration of all proteins to be tested was adjusted to 0.5 mg/mL with PBS.

(2) 100 μL of various protein solutions and blank PBS solution as a control were added to the 96-well plate and let stand at room temperature for 60 min.

(3) 10⁵ 3T3 cells in good culture status were added to each well and incubated at 37° C. for 60 min.

(4) Each well was washed 4 times with PBS.

(5) The absorbance at OD492 nm was detected using an LDH detection kit (Roche, 04744926001). The cell adhesion rate could be calculated based on the value of the blank control. The calculation formula was as follows: cell adhesion

$$rate = \frac{test well - bank well}{positive well - blank well} \times 100 \% .$$

The cell adhesion rate could reflect the activity of the collagen. The higher the activity of the protein, the better it can provide cells with a high-quality external environment in a short time, helping the adhesion of cells.

The results were shown in FIG. 8. From the comparison, it could be seen that the recombinant type II humanized collagen of the present disclosure had better bioadhesion activity compared with the bovine type I collagen (B col I), that is, the recombinant type II humanized collagen HC2B-A10>the bovine type I collagen. Surprisingly, the present inventors had demonstrated that for HC2B-A10, eight repeating sequences could ensure that the molecular weight of the recombinant type II humanized collagen was easy to purify and stable, and had excellent bioadhesion activity.

### Example 3: Mass Spectrometry Detection of the Recombinant Type II Humanized Collagen HC2B-A10

### Experimental Method

| | Matrix-assisted laser desorption ionization-time | | |
|---|---|---|---|
| | of flight mass spectrometer MALDI-TOF/TOF | | |
| Instrument name | Ultraflextreme ™, Brucker, Germany | | |
| Matrix | CHCA | Laser energy | 125 |
| Data retrieval | Mascot | Retrieval | ALL entries |
| software | | species | |

| | |
|---|---|
| Retrieval database | NCBIprot |

The recombinant type II humanized collagen HC2B-A10 with good purification and enzymatic digestion effects provided by the present disclosure was subjected to mass spectrometry detection, and the specific process was as follows:
the protein samples were reduced with DTT and alkylated with iodoacetamide followed by adding the trypsin to enzymatic digestion overnight. The peptide fragments obtained after enzyme digestion were desalted by C18ZipTip, mixed with the matrix α-cyano-4-hydroxycinnamic acid (CHCA) and plated. Finally, the matrix-assisted laser desorption ionization-time of flight mass spectrometer MALDI-TOF/TOF Ulraflextreme™, Brucker, Germany was used for analysis (See Protein J. 2016; 35:212-7 for peptide fingerprinting technology).

Data retrieval was handled by the MS/MS Ion Search page from the local Masco website. The protein identification results were obtained based on the primary mass spectrometry of the peptide fragments produced after enzymatic digestion. Detection parameters: Trypsin enzymatic digestion, with two missed cleavage sites. The alkylation of cysteine was set as a fixed modification. The oxidation of methionine was a variable modification. The database used for identification was NCBprot.

TABLE 1
Molecular weight and corresponding peptides
detected by mass spectrometry of HC2B-A10

| | Mr | |
|---|---|---|
| | (expected | |
| Observations | value) | peptide |
| 825.4722 | 824.4650 | GTPGLPGVK |
| | | (SEQ ID NO: 31) |
| 1175.6411 | 1174.6338 | GTPGLPGVKGHR |
| | | (SEQ ID NO: 32) |
| 877.4276 | 876.4203 | GYPGLDGAK |
| | | (SEQ ID NO: 33) |
| 2034.0504 | 2033.0432 | GYPGLDGAKGTPGLPGVK |
| | | GHR (SEQ ID NO: 34) |

The coverage rate of the detected peptide
functional region was 100%
GTPGLPGVKGHRGYPGLDGAKGTPGLPGVKGHRGYPGLDGAKGTP
GLPGVKGHRGYPGLDGAKGTPGLPGVKGHRGYPGLDGAKGTPGLP
GVKGHRGYPGLDGAKGTPGLPGVKGHRGYPGLDGAKGTPGLPGVK
GHRGYPGLDGAKGTPGLPGVKGHRGYPGLDGAKGTPGLPGVKGHR
GYPGLDGAKGTPGLPGVKGHRGYPGLDGAKGTPGLPGVKGHRGYP
GLDGAKGTPGLPGVKGHRGYPGLDGAKGTPGLPGVKGHRGYPGLD
GAKGTPGLPGVKGHRGYPGLDGAKGTPGLPGVKGHRGYPGLDGAK
GTPGLPGVKGHRGYPGLDGAK
(SEQ ID NO: 30)
The DNA sequence of HC2B-a3 (SEQ ID NO: 16):
GGTAAACCAGGAGATGATGGAGAAGCAGGAAAACCAGGAAAAGCA
GGAGAAAGAGGACCGCCTGGACCGCAAGGAGCACGTGGATTTCCA
GGAACCCCGGGACTGCCGGGTGTGAAAGGTCATAGAGGATATCCG
GGACTGGATGGAGCAAAAGGAGAAGCAGGGGCACCGGGAGTTAAA
GGTGAGAGCGGAAGCCCGGGAGAAAATGGAAGCCCTGGTCCGATG
GGTCCGAGAGGGCTGCCGGGTGAAAGAGGGCGTACCGGTCCGGCA
GGAGCAGCAGGTGCAAGAGGAAATGATGGACAGCCG
The DNA sequence of HC2B-a6 (SEQ ID NO: 17):
GGAAAACCCGGGAAGGCCGGTGAGCGCGGTCCACCGGGTCCGCAG
GGCGCGCGTGGGTTCCCGGGCACCCCGGGTCTGCCGGGTGTTAAA
GGTCATCGTGGCAAGCCGGGCAAGGCTGGCGAGCGCGGTCCGCCA
GGTCCGCAAGGTGCGAGAGGCTTTCCGGGCACTCCGGGTTTGCCG
GGTGTCAAAGGTCACCGTGGTAAACCGGGCAAGGCGGGTGAGCGT
GGCCCACCGGGTCCGCAGGGTGCGCGTGGTTTTCCGGGCACGCCG
GGTTTGCCGGGCGTTAAAGGCCACCGCGGCAAACCGGGCAAGGCG
GGTGAACGTGGCCCACCGGGTCCGCAAGGTGCACGTGGCTTCCCG
GGCACCCCGGGTCTGCCTGGCGTGAAAGGTCACCGTGGCAAGCCG
GGCAAAGCTGGTGAACGTGGTCCGCCCGGTCCGCAGGGCGCGCGT
GGCTTTCCGGGAACCCCGGGCCTGCCGGGCGTTAAGGGTCATCGT
GGCAAACCGGGCAAGGCTGGTGAGCGCGGGCCACCGGGTCCTCAA
GGTGCCCGTGGCTTCCCGGGCACCCCGGGTCTGCCGGGTGTGAAG
GGTCACCGCGGTAAACCGGGCAAGGCAGGCGAACGCGG
The DNA sequence of HC2B-b1 (SEQ ID NO: 18):
GTGAACCAGGTCGTGAAGGTAGCCCAGGTGCAGATGGACCACCAGG
TCGTGATGGTGCAGCAGGAGTGAAAGGAGATCGTGGTGAAACCGGT
GCAGTAGGTGCACCTGGTGCGCCAGGTCCGCCAGGTAGCCCTGGT
CCAGCCGGTCCTACCGGAAAACAAGGGGATAGAGGAGAAGCAGGA
GCACAGGGTCCGATGGGTCCGTCAGGTCCGGCGGGTGCACGTGGT
ATTCAGGGTCCGCAGGGTCCGCGTGGTGATAAAGGTGAAGCAGGT
GAACCGGGGGAAAGAGGATTAAAAGGGCATCGTGGTTTTACGGGT
CTGCAGGGTCTGCCTGGTCCGCCTGGTCCGAGCGGTGATCAGGGT
GCAAGCGGTCCGGCAGGTCCGAGCGGACCTCGTGGACCTCCGGGT
CCTGTGGGTCCTAGTGGTAAGGATGGGGCAAATGGTATTCCTGGT
CCTATTGGTCCGCCGGGTCCGCGTGGGAGATCAGGTGAAACCGGA
CCGGCA
The DNA sequence of HC2B-b3 (SEQ ID NO: 19):
GGTAGCCCAGGTCCAGCAGGTCCGACAGGTAAACAAGGAGATCGT
GGTGAAGCAGGAGCACAAGGACCAATGGGTCCAAGCGGTCCGGCA
GGTGCAAGAGGTATTCAAGGGCCGCAAGGGCCACGCGGTGATAAA
GGGGAAGCAGGTGAACCAGGTGAGAGAGGGTTAAAAGGACATCGT
GGATTTACAGGACTGCAGGGTTTACCAGGTCCGCCGGGACCGAGC
GGAGATCAAGGTGCAAGCGGTCCGGCGGGTCCGAGTGGTCCTCGT
The DNA sequence of HC2B-b5 (SEQ ID NO: 20):
GGAGCTAGGGGTATCCAGGGTCCGCAGGGCCCGCGGGGAGATAAA
GGGGAAGCGGGTGAACCGGGTGAACGTGGCTTGAAGGGTCATCGT
GGCTTCACCGGCCTGCAGGGCTTGCCGGGTCCGCCGGGTCCGTCC
GGTGCCCGTGGCATCCAGGGACCGCAAGGACCGCGTGGAGACAAA
GGTGAAGCGGGTGAACCGGGTGAGCGCGGCCTCAAGGGCCACCGC
GGCTTCACCGGTTTGCAAGGCCTGCCGGGTCCGCCGGGTCCGTCG
GGCGCGCGTGGTATTCAAGGCCCGCAAGGTCCACGCGGCGACAAA
GGCGAAGCGGGTGAGCCGGGCGAACGCGGCCTGAAAGGTCACCGC
GGTTTTACGGGTCTGCAGGGCCTGCCGGGTCCACCGGGTCCGAGC
GGTGCTCGCGGCATCCAGGGCCCGCAAGGTCCGCGTGGTGATAAG
GGTGAGGCGGGTGAGCCGGGAGAACGTGGTCTGAAGGGTCATAGA
GGCTTCACCGGTCTGCAAGGTCTGCCGGGTCCACCGGGCCCGAGC
GGCGCTAGGGGCATTCAGGGCCCGCAGGGGCCGCGTGGTGATAAG
GGCGAGGCCGGTGAGCCGGGCGAACGTGGTTTAAAAGGTCATCGC
GGCTTCACGGGCCTCCAAGGTCTGCCTGGCCCACCGGGTCCGAGC
GGCGCGCGTGGCATTCAAGGCCCACAGGGTCCTCGTGGCGACAAA
GGCGAGGCAGGCGAGCCGGGTGAGCGCGGTCTGAAAGGGCACCGT
GGTTTTACCGGTTTGCAAGGCCTGCCGGGCCCACCGGGCCCGTCC
GGTGCACGTGGCATTCAGGGTCCGCAGGGTCCGCGTGGCGACAAG
GGCGAAGCTGGTGAGCCGGGTGAGCGCGGCCTTAAGGGTCACCGT
GGCTTTACCGGTTTACAAGGTTTGCCGGGCCCTCCGGGTCCAAGC
GGTGCACGTGGTATCCAAGGCCCACAGGGTCCGCGTGGGGATAAA
GGCGAAGCGGGTGAACCGGGCGAGCGCGGGCTGAAGGGTCACCGT
GGCTTTACTGGCCTGCAGGGTCTGCCGGGTCCACCGGGTCCGTCT
The DNA sequence of B3Q (SEQ ID NO: 21):
GGATCACCCGGGCCGGCGGGCCCGACCGGTAAACAGGGTGACCGC
GGTGAGGCGGGTGCGCAGGGGCCGATGGGTCCGAGCGGTCCGGCT
GGTGCGCGTGGTATCCAGGGACCGCAGGGCCCACGCGGTGATAAA
GGCGAAGCAGGCGAACCGGGTGAGCGCGGTTTGAAGGGCCACCGT
GGTTTTACCGGTCTGCAGGGCCTGCCGGGCCCGCCGGGTCCGTCA
GGCGATCAGGGTGCTAGCGGTCCGGCTGGCCCGTCCGGCCCGCGC
GGTAGCCCGGGCCCAGCTGGCCCGACTGGTAAACAGGGCGACCGT
GGCGAGGCCGGTGCCCAAGGCCCTATGGGCCCGAGCGGTCCGGCA
GGCGCGCGGGGCATCCAGGGTCCGCAAGGACCAAGAGGCGACAAG
GGTGAGGCAGGCGAGCCTGGCGAACGTGGCCTGAAGGGTCACCGC
GGTTTTACCGGTCTCCAAGGTCTGCCTGGTCCACCGGGTCCGTCT
GGAGATCAAGGCGCCAGCGGCCCCGCGGGTCCCTCCGGTCCGCGT
GGCAGCCCGGGTCCGGCAGGACCAACGGGTAAGCAGGGTGATAGA
GGCGAAGCCGGTGCGCAGGGCCCGATGGGTCCTTCGGGTCCAGCG
GGGGCGCGTGGCATTCAAGGTCCGCAGGGCCCGCGTGGTGACAAA
GGTGAGGCGGGCGAACCGGGCGAGCGCGGCTTGAAGGGTCATCGT
GGATTCACCGGTTTGCAAGGCCTGCCGGGCCCCCCGGGCCCGAGC
GGTGACCAAGGAGCTAGCGGGCCGGCTGGTCCGTCGGGCCCGCGT
GGCAGTCCGGGTCCGGCGGGCCCAACGGGCAAGCAAGGAGATCGT
GGTGAGGCGGGTGCGCAAGGTCCGATGGGTCCGTCTGGTCCGGCG
GGTGCACGCGGTATTCAAGGTCCGCAGGGGCCGCGTGGTGATAAA
GGAGAAGCAGGCGAACCGGGTGAACGTGGTCTGAAAGGTCATCGT
GGCTTCACCGGCCTGCAGGGTTTACCAGGTCCACCGGGTCCGTCT
GGCGACCAGGGTGCCAGCGGTCCGGCGGGCCCGTCCGGACCGCGT
TAA
The DNA sequence of HC2B-A7 (SEQ ID NO: 22):
GGACCCCAAGGGGCGCGTGGTTTTCCGGGCACCCCGGGCCTGCCG
GGCGTGAAGGGTCACCGCGGTTATCCGGGGCTGGATGGCGCGAAG
GGTCCGCAAGGTGCTCGCGGTTTCCCGGGAACCCCGGGCCTGCCG
GGCGTTAAAGGTCATCGTGGTTATCCGGGTCTGGATGGTGCGAAG
GGCCCACAGGGTGCGCGTGGTTTCCCGGGCACCCCTGGGCTCCCG
GGTGTGAAAGGTCACAGAGGTTACCCGGGCCTGGATGGTGCCAAA
GGTCCGCAAGGTGCGCGTGGTTTTCCGGGCACGCCGGGCTTGCCG
GGTGTTAAAGGTCACCGTGGTTATCCGGGTCTGGACGGCGCAAAG
GGTCCTCAGGGTGCACGCGGCTTCCCGGGTACGCCGGGCCTTCCG
GGCGTGAAGGGCCACCGCGGTTATCCGGGTTTGGACGGTGCCAAG
GGTCCGCAGGGTGCGCGTGGATTCCCGGGAACCCCAGGCCTGCCA
GGTGTGAAGGGCCACCGTGGCTACCCGGGCTTGGACGGTGCTAAA
GGACCGCAGGGCGCACGTGGCTTTCCGGGTACCCCGGGCCTGCCT
GGCGTCAAGGGCCATCGTGGTTACCCGGGCCTGGATGGTGCAAAA
GGCCCACAGGGTGCCCGTGGTTTCCCGGGTACTCCGGGCCTGCCG
GGCGTAAAAGGCCATCGCGGTTACCCGGGTTTGGATGGCGCTAAA
GGCCCACAAGGTGCTAGAGGGTTCCCTGGCACCCCGGGTCTGCCG
GGCGTTAAAGGCCATCGTGGTTACCCGGGCTTGGACGGCGCGAAG
GGTCCGCAAGGCGCGCGTGGTTTTCCGGGTACCCCGGGTCTGCCA
GGCGTTAAGGGTCACCGCGGTTACCCGGGTTTAGACGGTGCGAAA
The DNA sequence of HC2B-A8 (SEQ ID NO: 23):
GGTGCTAGGGGATTCCCGGGAACCCCGGGTCTGCCAGGCGTGAAA
GGTCACCGCGGTTACCCGGGCCTCGACGGCGCGAAGGGTGCCCGT
GGTTTTCCGGGAACCCCGGGCTTGCCAGGTGTCAAGGGCCATCGT
GGTTACCCGGGTCTCGATGGTGCAAAGGGTGCGAGAGGCTTCCCG
GGCACCCCGGGCCTGCCAGGGGTGAAAGGCCACAGAGGCTATCCT
GGCTTGGATGGTGCCAAGGGTGCACGTGGATTCCCGGGCACTCCG
GGTCTGCCGGGCGTGAAGGGCCACCGCGGTTATCCGGGCCTGGAC
GGTGCTAAAGGCGCGCGTGGTTTTCCGGGTACGCCGGGCTTGCCA
GGTGTTAAGGGCCACCGTGGCTACCCGGGGCTGGATGGTGCCAAA
GGTGCTCGCGGTTTCCCGGGAACCCCGGGTCTGCCTGGCGTGAAG
GGTCATCGTGGTTACCCGGGCTTGGACGGCGCTAAGGGTGCGCGT
GGTTTTCCGGGCACCCCGGGTCTGCCGGGGGTGAAAGGTCACCGC
GGTTATCCCGGTCTGGATGGTGCGAAGGGTGCGCGTGGCTTCCCG
GGCACCCCGGGCCTGCCGGGTGTTAAAGGTCATCGTGGTTACCCG
GGCCTGGATGGTGCCAAGGGCGCTCGCGGTTTTCCGGGCACGCCA
GGTTTACCGGGGGTCAAAGGCCATCGTGGCTATCCGGGTTTAGAT
GGCGCGAAAGGCGCACGCGGATTCCCGGGAACCCCGGGCCTGCCT
GGCGTTAAAGGCCACCGCGGTTACCCGGGCCTTGACGGCGCGAAA
GGCGCGCGTGGTTTTCCGGGCACCCCGGGTCTGCCGGGTGTTAAA
GGTCACCGTGGCTATCCGGGTCTGGACGGTGCAAAAGGTGCACGT
GGTTTCCCGGGGACTCCGGGCCTGCCGGGTGTTAAGGGCCATCGT
GGTTACCCGGGTTTGGACGGTGCGAAG
The DNA sequence of HC2B-A9 (SEQ ID NO: 24):
GGATTTCCCGGGACTCCAGGTCTGCCCGGCGTGAAGGGCCACCGC
GGTTATCCGGGCTTGGACGGTGCGAAAGGCTTCCCGGGCACGCCA
GGCTTACCTGGTGTGAAGGGCCATCGTGGTTACCCGGGGCTGGAT
GGTGCGAAGGGTTTTCCGGGCACCCCGGGTCTGCCGGGCGTAAAG
GGCCACCGCGGTTATCCGGGCCTGGACGGTGCAAAAGGTTTTCCG
GGCACCCCGGGCCTGCCGGGTGTGAAGGGCCACCGCGGTTACCCG
GGTTTGGACGGTGCCAAGGGATTCCCAGGTACGCCGGGTCTGCCG
GGGGTGAAGGGCCATAGAGGCTACCCGGGCTTGGATGGTGCGAAA
GGTTTCCCGGGAACCCCTGGTCTGCCGGGCGTTAAGGGCCACCGT
GGTTATCCGGGTCTGGATGGCGCGAAAGGTTTTCCGGGAACCCCA
GGTCTGCCGGGCGTTAAAGGTCATCGTGGTTACCCGGGGCTCGAT
GGTGCCAAAGGTTTTCCGGGCACCCCGGGTCTGCCGGGCGTCAAA
GGTCACCGCGGTTATCCGGGTCTGGATGGTGCGAAAGGCTTCCCG
GGAACCCCGGGCCTGCCTGGCGTGAAAGGCCATCGTGGTTATCCG
GGTCTGGATGGTGCTAAAGGATTCCCGGGAACCCCGGGCCTGCCG
GGCGTTAAGGGTCACCGTGGTTACCCGGGCCTGGATGGCGCTAAG
GGTTTCCCGGGTACGCCCGGCCTGCCGGGCGTCAAAGGTCATCGT
GGTTACCCGGGTTTAGACGGCGCAAAAGGTTTCCCGGGAACCCCG
GGTCTGCCGGGTGTTAAGGGCCATCGTGGCTATCCGGGCTTGGAC
GGTGCAAAGGGTTTTCCTGGTACTCCAGGCTTGCCCGGCGTTAAG
GGCCACCGTGGTTACCCGGGTTTGGACGGCGCTAAAGGCTTTCCG
GGAACCCCGGGTCTTCCGGGCGTGAAAGGTCACCGTGGTTACCCG
GGTCTGGACGGCGCGAAG
The DNA sequence of HC2B-A10 (SEQ ID NO: 25):
GGAACACCCGGGCTGCCGGGTGTTAAGGGCCATCGTGGCTACCCG
GGTTTGGACGGTGCGAAAGGCACCCCTGGCCTGCCGGGTGTCAAA
GGCCATCGTGGTTATCCGGGTCTCGACGGTGCGAAGGGTACCCCA
GGACTGCCGGGTGTTAAAGGTCACCGCGGTTACCCGGGTCTGGAC
GGCGCAAAAGGTACTCCAGGCCTGCCGGGCGTGAAGGGTCATCGT
GGTTACCCGGGCCTGGACGGCGCGAAGGGCACCCCGGGCTTACCG
GGCGTGAAAGGCCATCGTGGTTATCCGGGGTTGGATGGCGCAAAG
GGTACCCCGGGTCTGCCTGGTGTTAAGGGCCACCGCGGTTATCCG
GGCCTGGATGGTGCGAAAGGTACGCCGGGATTGCCGGGTGTGAAG
GGTCATCGTGGTTACCCGGGTCTGGATGGTGCCAAAGGCACGCCA
GGCCTTCCGGGCGTCAAGGGCCACCGCGGTTACCCGGGCCTGGAT
GGTGCGAAAGGTACCCCGGGTTTGCCTGGTGTTAAGGGCCACCGC
GGTTATCCTGGTTTGGACGGCGCGAAAGGAACCCCGGGCCTGCCG
GGTGTTAAAGGCCATCGTGGCTACCCGGGTCTGGACGGCGCTAAA
GGCACTCCGGGTCTGCCGGGCGTGAAGGGCCACCGTGGTTATCCG
GGTTTAGACGGCGCGAAAGGCACCCCAGGCCTGCCGGGTGTCAAG
GGTCACCGCGGTTACCCGGGTCTGGATGGTGCCAAGGGCACCCCG
GGCTTGCCGGGCGTGAAAGGACATCGTGGTTACCCGGGCCTGGAT
GGTGCTAAGGGCACGCCAGGTCTTCCGGGTGTGAAAGGTCACCGT
GGTTATCCGGGTCTGGACGGTGCAAAGGGAACCCCGGGGCTGCCG
GGCGTAAAAGGCCACAGAGGTTATCCGGGTCTTGATGGTGCGAAG
GGCACCCCGGGCCTGCCGGGCGTTAAAGGTCACCGTGGTTACCCG
GGTTTGGACGGCGCTAAA
The DNA sequence of HC2B-B7 (SEQ ID NO: 26):
GGAGAAAGGGGGTTGAAGGGACACCGCGGTTTTACTGGTTTGCAA
GGCCTGCCGGGCCCTCCGGGTCCGTCTGGCGATCAGGGTGCAAGC
GGCCCGGCGGGTCCGTCGGGCCCGCGTGGTGAGCGCGGTCTTAAG
GGCCATCGTGGTTTCACCGGTTTACAAGGTCTGCCGGGCCCGCCG
GGTCCGAGCGGTGATCAAGGGGCCTCCGGTCCGGCTGGCCCGTCC
GGCCCAAGAGGCGAACGTGGTCTGAAAGGTCATCGTGGATTCACC
GGACTGCAGGGTCTGCCTGGTCCGCCTGGTCCGTCAGGCGACCAA
GGTGCGAGCGGTCCGGCGGGTCCGTCCGGTCCGCGTGGTGAACGT
GGTCTGAAAGGCCACCGCGGCTTCACCGGTTTGCAAGGCCTGCCA
GGCCCACCGGGTCCGTCTGGCGACCAGGGAGCCAGCGGTCCGGCT
GGCCCATCTGGCCCACGCGGCGAGCGCGGTCTGAAAGGCCACCGT
GGCTTTACGGGCTTGCAGGGTCTCCCGGGCCCACCGGGCCCGAGC
GGTGATCAGGGTGCCAGCGGACCGGCAGGCCCCTCTGGTCCGCGT
GGTGAACGTGGCCTGAAAGGTCATCGTGGTTTTACCGGTTTACAG
GGCCTGCCAGGTCCCCCGGGTCCGTCCGGCGACCAGGGCGCAAGC
GGTCCGGCTGGCCCGAGCGGTCCGCGTGGCGAGCGCGGCCTTAAG
GGCCACAGAGGCTTCACGGGTCTGCAAGGTTTGCCGGGTCCGCCT
GGCCCGTCGGGCGATCAGGGCGCGAGCGGCCCGGCGGGTCCGAGC
GGTCCGCGTGGCGAGCGTGGTCTGAAGGGTCACCGCGGTTTTACC
GGTCTGCAAGGTCTGCCGGGTCCGCCTGGCCCGAGCGGCGACCAG
GGAGCGAGCGGTCCGGCGGGTCCGAGTGGTCCGCGT
The DNA sequence of HC2B-B8 (SEQ ID NO: 27):
GGGGAAAGGGGACTCAAAGGTCACCGCGGTTTCACGGGCCTTCAA
GGTCTGCCGGGTCCTCCGGGTCCGAGCGGCGACCAAGGTGCGTCT
GGCCCAGCGGGTGAGCGTGGTTTAAAAGGCCACCGCGGTTTCACC
GGCCTGCAGGGTTTACCGGGTCCGCCTGGCCCGAGCGGTGATCAA
GGTGCAAGCGGCCCGGCAGGCGAACGCGGTCTGAAAGGCCATAGA
GGTTTTACCGGCCTGCAGGGCTTGCCGGGCCCGCCGGGCCCGAGT
GGCGATCAAGGTGCTTCCGGCCCGGCGGGTGAACGTGGCCTGAAG
GGCCATCGTGGCTTTACCGGTCTGCAGGGCCTGCCAGGTCCGCCG
GGCCCGTCTGGCGACCAGGGTGCGAGCGGTCCAGCCGGTGAGCGC
GGCTTGAAGGGCCACCGCGGCTTTACGGGTTTGCAAGGTCTGCCT
GGTCCGCCGGGCCCGTCAGGCGATCAAGGGGCGAGCGGCCCGGCG
GGTGAACGTGGTCTGAAAGGTCATCGTGGATTCACCGGCCTGCAG
GGTCTGCCGGGCCCGCCCGGCCCGTCCGGTGACCAGGGTGCTTCC
GGTCCGGCTGGTGAGCGCGGTCTTAAGGGACACCGTGGCTTCACC
GGTCTGCAGGGCTTGCCGGGTCCTCCGGGGCCGAGCGGCGACCAG
GGTGCGTCCGGCCCGGCGGGTGAACGTGGTCTGAAGGGCCACCGT
GGTTTTACCGGCTTGCAAGGTCTGCCGGGTCCACCGGGTCCGTCT
GGCGATCAGGGAGCCAGCGGTCCGGCAGGCGAGCGTGGTTTGAAG
GGTCACCGTGGATTCACCGGCCTGCAGGGGCTGCCGGGCCCGCCG
GGTCCGTCGGGCGATCAGGGAGCGAGCGGTCCGGCAGGTGAGCGT
GGTCTGAAAGGCCATCGTGGCTTCACTGGTTTGCAAGGCCTGCCT
GGCCCACCGGGTCCGAGCGGTGACCAGGGTGCCAGCGGTCCGGCT
The DNA sequence of HC2B-B9 (SEQ ID NO: 28):
GGAGAAAGGGGGCTTAAGGGCCATCGTGGCTTCACGGGTTTGCAG
GGCCTGCCGGGCCCACCGGGTCCGAGCGGCGATCAAGGCGAGCGG
GGCCTGAAAGGTCATCGTGGCTTTACCGGTTTGCAGGGCCTGCCG
GGCCCACCGGGACCGAGCGGTGATCAGGGGGAGCGTGGTCTGAAA
GGTCATCGTGGTTTTACCGGTCTGCAGGGTTTGCCGGGTCCTCCG
GGTCCGTCTGGCGACCAAGGTGAGCGTGGTCTGAAAGGTCATCGT
GGTTTCACCGGTTTACAAGGCCTGCCGGGCCCACCGGGTCCGAGC
GGTGACCAAGGCGAGCGTGGCCTGAAAGGCCACCGCGGTTTCACC
GGTCTTCAGGGTCTGCCGGGCCCACCGGGTCCCAGCGGTGACCAG
GGTGAACGCGGTCTGAAGGGTCACAGAGGCTTCACCGGTCTCCAA
GGCTTACCGGGTCCGCCGGGCCCGAGCGGTGATCAGGGCGAACGT
GGTCTGAAGGGCCACCGTGGCTTTACGGGTCTGCAAGGCCTGCCG
GGTCCGCCGGGTCCGTCGGGCGACCAGGGTGAGCGCGGTTTGAAG
GGCCACCGTGGATTCACCGGCCTGCAGGGCCTGCCGGGACCGCCT
GGTCCGTCAGGTGATCAGGGTGAGCGCGGTCTCAAGGGTCATCGT
GGTTTTACCGGTTTACAAGGTTTGCCGGGTCCGCCTGGCCCGTCT
GGTGATCAAGGCGAACGTGGTCTGAAAGGCCACCGCGGCTTCACT
GGTCTGCAGGGCTTGCCGGGCCCACCGGGTCCGTCCGGCGATCAA
GGCGAACGTGGTCTGAAGGGTCACCGCGGATTCACGGGCCTGCAG
GGCCTGCCGGGTCCGCCAGGTCCGTCCGGCGACCAGGGCGAACGC
GGGCTGAAAGGTCACCGTGGCTTTACCGGTTTGCAAGGACTGCCG
GGTCCGCCGGGCCCGAGCGGCGACCAG
The DNA sequence of HC2B-B10 (SEQ ID NO: 29):
GGAGAAAGGGGGCTGAAGGGTCACCGTGGTTTTACCGGTTTGCAA
GGTTTGCCGGGTCCGCCGGGTGAGCGCGGCCTGAAGGGTCATCGT
GGCTTCACCGGCCTGCAAGGCCTGCCGGGTCCGCCGGGTGAGCGT
GGTCTCAAGGGTCATCGTGGCTTTACCGGTTTACAGGGTCTGCCG
GGCCCACCGGGCGAGCGCGGTCTGAAAGGCCATCGTGGATTCACC
GGCCTGCAGGGCTTGCCGGGTCCACCGGGCGAAAGAGGCTTAAAA
GGTCACCGTGGTTTTACGGGTCTGCAAGGTCTGCCGGGTCCGCCG
GGTGAACGTGGCCTTAAGGGTCACCGCGGCTTCACTGGTCTGCAG
GGTTTGCCGGGTCCGCCTGGTGAAAGGGGTCTGAAAGGCCATCGT
GGTTTCACCGGTCTGCAGGGCCTGCCGGGCCCACCGGGTGAGAGA
GGCTTGAAGGGTCACCGTGGTTTTACGGGCCTGCAGGGTCTGCCG
GGCCCGCCGGGTGAGCGCGGTCTTAAGGGCCACCGTGGATTCACC
GGCCTTCAAGGTCTGCCGGGTCCGCCGGGGGAGCGTGGCCTGAAA
GGTCATCGCGGCTTCACCGGTTTGCAAGGTTTGCCAGGCCCACCG
GGCGAACGTGGCCTGAAAGGGCACCGTGGTTTTACTGGTTTGCAG
GGCCTGCCGGGCCCGCCGGGCGAGCGCGGTTTGAAGGGCCACCGT
GGTTTCACGGGCCTGCAAGGTCTGCCGGGCCCTCCGGGAGAACGC
GGTCTGAAAGGTCACCGTGGCTTCACCGGCCTGCAGGGTCTACCG
GGTCCACCGGGCGAGCGCGGACTGAAAGGTCATCGCGGCTTCACC
GGTCTGCAGGGCCTGCCGGGTCCGCCGGGTGAACGTGGCCTGAAA
GGCCACCGCGGTTTTACCGGTTTACAAGGCTTGCCGGGTCCGCCG
GGTGAACGTGGCTTGAAGGGCCATCGTGGCTTTACCGGTCTTCAG
GGGCTGCCGGGCCCGCCT

1. A collagen comprising a plurality of repeating units, wherein the repeating unit comprises an amino acid sequence as shown in SEQ ID NO: 15, an amino acid sequence obtained by a substitution, insertion, deletion or addition of one or more amino acid residues of the amino acid sequence of SEQ ID NO: 15, or an amino acid sequence having 80% to 99% identity thereto, and each of the repeating units is directly connected or separated by one or more amino acid residues, the number of the repeating unit is 2 or more, the collagen is a recombinant type II humanized collagen.

2. A polynucleotide encoding the collagen according to claim 1.

3. A nucleic acid comprising the polynucleotide according to claim 2, optionally further comprising nucleotides encoding a purification tag, and optionally further comprising nucleotides encoding a leader sequence.

4. A vector comprising the polynucleotide according to claim 2.

5. A host cell comprising the polynucleotide according to claim 2.

6. A method for producing the collagen according to claim 1, comprising:
(1) culturing a host cell under a suitable culture condition;
(2) harvesting the host cells and/or culture medium containing the collagen; and
(3) purifying the collagen.

7. A composition comprising the collagen according to claim 1.

8. The composition according to claim 7, being the composition for cartilage repair.

9. A method for increasing cell adhesion, comprising contacting the cells with the collagen according to claim 1.

10. A method for preparing one or more of a biological dressing, a human bionic material, a plastic surgery material, an organoid culture material, a cardiovascular stent material, a coating material, a tissue injection filling material, an ophthalmic material, an obstetrics and gynecology biomaterial, a nerve repair regenerative material, a liver tissue material and blood vessel repair regeneration material, a 3D printed artificial organ biomaterial, a cosmetic raw material, a pharmaceutical excipient, and a food additive, including using the collagen according to claim 1.

11. A method for cartilage repair, including using the collagen according to claim 1.

12. The collagen of claim 1, wherein the number of the repeating unit is 6-10.

13. The collagen of claim 1, wherein the collagen comprises an amino acid sequence as shown in SEQ ID NO: 10, an amino acid sequence obtained by a substitution, insertion, deletion or addition of one or more amino acid residues of the amino acid sequence of SEQ ID NO: 10, or a variant having 80% to 99% identity thereto.

14. The polynucleotide of claim 2, comprising the nucleotide sequence as shown in SEQ ID NO: 29.

15. The nucleic acid of claim 3, wherein the purification tag is a His tag, a GST tag, an MBP tag, a SUMO tag, or a NusA tag.

16. The vector of claim 4, wherein the vector is an expression vector.

17. The vector of claim 16, wherein the vector comprises an expression control element operably linked to the polynucleotide or the nucleic acid.

18. The vector of claim 17, wherein the expression control element is a promoter, a terminator and/or an enhancer.

19. The host cell of claim 5, which is a bacterium, a fungus, or an animal cell.

20. The composition of claim 8, which is one or more of a biological dressing, a human bionic material, a plastic surgery material, an organoid culture material, a cardiovascular stent material, a coating material, a tissue injection filling material, an ophthalmic material, an obstetrics and gynecology biomaterial, a nerve repair regenerative material, a liver tissue material, and blood vessel repair regeneration material, a 3D printed artificial organ biomaterial, a cosmetic raw material, a pharmaceutical excipient, and a food additive and/or which is an injectable composition or an oral composition.


SEQUENCE LISTING (34 sequences)

SEQ ID NO: 1 - protein, 102 aa, synthetic construct
GKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQP

SEQ ID NO: 2 - protein, 208 aa, synthetic construct
GKPGKAGERGPPGPQGARGFPGTPGLPGVKGHRGKPGKAGERGPPGPQGARGFPGTPGLPGVKGHRGKPGKAGERGPPGPQGARGFPGTPGLPGVKGHRGKPGKAGERGPPGPQGARGFPGTPGLPGVKGHRGKPGKAGERGPPGPQGARGFPGTPGLPGVKGHRGKPGKAGERGPPGPQGARGFPGTPGLPGVKGHRGKPGKAGERG

SEQ ID NO: 3 - protein, 168 aa, synthetic construct
GEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPA

SEQ ID NO: 4 - protein, 90 aa, synthetic construct
GSPGPAGPTGKQGDRGEAGAQGPMGPSGPAGARGIQGPQGPRGDKGEAGEPGERGLKGHRGFTGLQGLPGPPGPSGDQGASGPAGPSGPR

SEQ ID NO: 5 - protein, 360 aa, synthetic construct
GARGIQGPQGPRGDKGEAGEPGERGLKGHRGFTGLQGLPGPPGPSGARGIQGPQGPRGDKGEAGEPGERGLKGHRGFTGLQGLPGPPGPSGARGIQGPQGPRGDKGEAGEPGERGLKGHRGFTGLQGLPGPPGPSGARGIQGPQGPRGDKGEAGEPGERGLKGHRGFTGLQGLPGPPGPSGARGIQGPQGPRGDKGEAGEPGERGLKGHRGFTGLQGLPGPPGPSGARGIQGPQGPRGDKGEAGEPGERGLKGHRGFTGLQGLPGPPGPSGARGIQGPQGPRGDKGEAGEPGERGLKGHRGFTGLQGLPGPPGPSGARGIQGPQGPRGDKGEAGEPGERGLKGHRGFTGLQGLPGPPGPS

SEQ ID NO: 6 - protein, 360 aa, synthetic construct
GSPGPAGPTGKQGDRGEAGAQGPMGPSGPAGARGIQGPQGPRGDKGEAGEPGERGLKGHRGFTGLQGLPGPPGPSGDQGASGPAGPSGPRGSPGPAGPTGKQGDRGEAGAQGPMGPSGPAGARGIQGPQGPRGDKGEAGEPGERGLKGHRGFTGLQGLPGPPGPSGDQGASGPAGPSGPRGSPGPAGPTGKQGDRGEAGAQGPMGPSGPAGARGIQGPQGPRGDKGEAGEPGERGLKGHRGFTGLQGLPGPPGPSGDQGASGPAGPSGPRGSPGPAGPTGKQGDRGEAGAQGPMGPSGPAGARGIQGPQGPRGDKGEAGEPGERGLKGHRGFTGLQGLPGPPGPSGDQGASGPAGPSGPR

SEQ ID NO: 7 - protein, 300 aa, synthetic construct
GPQGARGFPGTPGLPGVKGHRGYPGLDGAKGPQGARGFPGTPGLPGVKGHRGYPGLDGAKGPQGARGFPGTPGLPGVKGHRGYPGLDGAKGPQGARGFPGTPGLPGVKGHRGYPGLDGAKGPQGARGFPGTPGLPGVKGHRGYPGLDGAKGPQGARGFPGTPGLPGVKGHRGYPGLDGAKGPQGARGFPGTPGLPGVKGHRGYPGLDGAKGPQGARGFPGTPGLPGVKGHRGYPGLDGAKGPQGARGFPGTPGLPGVKGHRGYPGLDGAKGPQGARGFPGTPGLPGVKGHRGYPGLDGAK

SEQ ID NO: 8 - protein, 324 aa, synthetic construct
GARGFPGTPGLPGVKGHRGYPGLDGAKGARGFPGTPGLPGVKGHRGYPGLDGAKGARGFPGTPGLPGVKGHRGYPGLDGAKGARGFPGTPGLPGVKGHRGYPGLDGAKGARGFPGTPGLPGVKGHRGYPGLDGAKGARGFPGTPGLPGVKGHRGYPGLDGAKGARGFPGTPGLPGVKGHRGYPGLDGAKGARGFPGTPGLPGVKGHRGYPGLDGAKGARGFPGTPGLPGVKGHRGYPGLDGAKGARGFPGTPGLPGVKGHRGYPGLDGAKGARGFPGTPGLPGVKGHRGYPGLDGAKGARGFPGTPGLPGVKGHRGYPGLDGAK

SEQ ID NO: 9 - protein, 336 aa, synthetic construct
GFPGTPGLPGVKGHRGYPGLDGAKGFPGTPGLPGVKGHRGYPGLDGAKGFPGTPGLPGVKGHRGYPGLDGAKGFPGTPGLPGVKGHRGYPGLDGAKGFPGTPGLPGVKGHRGYPGLDGAKGFPGTPGLPGVKGHRGYPGLDGAKGFPGTPGLPGVKGHRGYPGLDGAKGFPGTPGLPGVKGHRGYPGLDGAKGFPGTPGLPGVKGHRGYPGLDGAKGFPGTPGLPGVKGHRGYPGLDGAKGFPGTPGLPGVKGHRGYPGLDGAKGFPGTPGLPGVKGHRGYPGLDGAKGFPGTPGLPGVKGHRGYPGLDGAKGFPGTPGLPGVKGHRGYPGLDGAK

SEQ ID NO: 10 - protein, 336 aa, synthetic construct
GTPGLPGVKGHRGYPGLDGAKGTPGLPGVKGHRGYPGLDGAKGTPGLPGVKGHRGYPGLDGAKGTPGLPGVKGHRGYPGLDGAKGTPGLPGVKGHRGYPGLDGAKGTPGLPGVKGHRGYPGLDGAKGTPGLPGVKGHRGYPGLDGAKGTPGLPGVKGHRGYPGLDGAKGTPGLPGVKGHRGYPGLDGAKGTPGLPGVKGHRGYPGLDGAKGTPGLPGVKGHRGYPGLDGAKGTPGLPGVKGHRGYPGLDGAKGTPGLPGVKGHRGYPGLDGAKGTPGLPGVKGHRGYPGLDGAKGTPGLPGVKGHRGYPGLDGAKGTPGLPGVKGHRGYPGLDGAK

SEQ ID NO: 11 - protein, 312 aa, synthetic construct
GERGLKGHRGFTGLQGLPGPPGPSGDQGASGPAGPSGPRGERGLKGHRGFTGLQGLPGPPGPSGDQGASGPAGPSGPRGERGLKGHRGFTGLQGLPGPPGPSGDQGASGPAGPSGPRGERGLKGHRGFTGLQGLPGPPGPSGDQGASGPAGPSGPRGERGLKGHRGFTGLQGLPGPPGPSGDQGASGPAGPSGPRGERGLKGHRGFTGLQGLPGPPGPSGDQGASGPAGPSGPRGERGLKGHRGFTGLQGLPGPPGPSGDQGASGPAGPSGPRGERGLKGHRGFTGLQGLPGPPGPSGDQGASGPAGPSGPR

SEQ ID NO: 12 - protein, 330 aa, synthetic construct
GERGLKGHRGFTGLQGLPGPPGPSGDQGASGPAGERGLKGHRGFTGLQGLPGPPGPSGDQGASGPAGERGLKGHRGFTGLQGLPGPPGPSGDQGASGPAGERGLKGHRGFTGLQGLPGPPGPSGDQGASGPAGERGLKGHRGFTGLQGLPGPPGPSGDQGASGPAGERGLKGHRGFTGLQGLPGPPGPSGDQGASGPAGERGLKGHRGFTGLQGLPGPPGPSGDQGASGPAGERGLKGHRGFTGLQGLPGPPGPSGDQGASGPAGERGLKGHRGFTGLQGLPGPPGPSGDQGASGPAGERGLKGHRGFTGLQGLPGPPGPSGDQGASGPA

SEQ ID NO: 13 - protein, 324 aa, synthetic construct
GERGLKGHRGFTGLQGLPGPPGPSGDQGERGLKGHRGFTGLQGLPGPPGPSGDQGERGLKGHRGFTGLQGLPGPPGPSGDQGERGLKGHRGFTGLQGLPGPPGPSGDQGERGLKGHRGFTGLQGLPGPPGPSGDQGERGLKGHRGFTGLQGLPGPPGPSGDQGERGLKGHRGFTGLQGLPGPPGPSGDQGERGLKGHRGFTGLQGLPGPPGPSGDQGERGLKGHRGFTGLQGLPGPPGPSGDQGERGLKGHRGFTGLQGLPGPPGPSGDQGERGLKGHRGFTGLQGLPGPPGPSGDQGERGLKGHRGFTGLQGLPGPPGPSGDQ

SEQ ID NO: 14 - protein, 336 aa, synthetic construct
GERGLKGHRGFTGLQGLPGPPGERGLKGHRGFTGLQGLPGPPGERGLKGHRGFTGLQGLPGPPGERGLKGHRGFTGLQGLPGPPGERGLKGHRGFTGLQGLPGPPGERGLKGHRGFTGLQGLPGPPGERGLKGHRGFTGLQGLPGPPGERGLKGHRGFTGLQGLPGPPGERGLKGHRGFTGLQGLPGPPGERGLKGHRGFTGLQGLPGPPGERGLKGHRGFTGLQGLPGPPGERGLKGHRGFTGLQGLPGPPGERGLKGHRGFTGLQGLPGPPGERGLKGHRGFTGLQGLPGPPGERGLKGHRGFTGLQGLPGPPGERGLKGHRGFTGLQGLPGPP

SEQ ID NO: 15 - protein, 42 aa, Homo sapiens
GTPGLPGVKGHRGYPGLDGAKGTPGLPGVKGHRGYPGLDGAK

SEQ ID NO: 16 - DNA, 306 bp, synthetic construct
  source 1..306: mol_type = other DNA
ggtaaaccaggagatgatggagaagcaggaaaaccaggaaaagcaggagaaagaggaccgcctggaccgcaaggagcacgtggatttccaggaaccccgggactgccgggtgtgaaaggtcatagaggatatccgggactggatggagcaaaaggagaagcaggggcaccgggagttaaaggtgagagcggaagcccgggagaaaatggaagccctggtccgatgggtccgagagggctgccgggtgaaagagggcgtaccggtccggcaggagcagcaggtgcaagaggaaatgatggacagccg

SEQ ID NO: 17 - DNA, 623 bp, synthetic construct
  source 1..623: mol_type = other DNA
ggaaaacccgggaaggccggtgagcgcggtccaccgggtccgcagggcgcgcgtgggttcccgggcaccccgggtctgccgggtgttaaaggtcatcgtggcaagccgggcaaggctggcgagcgcggtccgccaggtccgcaaggtgcgagaggctttccgggcactccgggtttgccgggtgtcaaaggtcaccgtggtaaaccgggcaaggcgggtgagcgtggcccaccgggtccgcagggtgcgcgtggttttccgggcacgccgggtttgccgggcgttaaaggccaccgcggcaaaccgggcaaggcgggtgaacgtggcccaccgggtccgcaaggtgcacgtggcttcccgggcaccccgggtctgcctggcgtgaaaggtcaccgtggcaagccgggcaaagctggtgaacgtggtccgcccggtccgcagggcgcgcgtggctttccgggaaccccgggcctgccgggcgttaagggtcatcgtggcaaaccgggcaaggctggtgagcgcgggccaccgggtcctcaaggtgcccgtggcttcccgggcaccccgggtctgccgggtgtgaagggtcaccgcggtaaaccgggcaaggcaggcgaacgcgg

SEQ ID NO: 18 - DNA, 503 bp, synthetic construct
  source 1..503: mol_type = other DNA
gtgaaccaggtcgtgaaggtagcccaggtgcagatggaccaccaggtcgtgatggtgcagcaggagtgaaaggagatcgtggtgaaaccggtgcagtaggtgcacctggtgcgccaggtccgccaggtagccctggtccagccggtcctaccggaaaacaaggggatagaggagaagcaggagcacagggtccgatgggtccgtcaggtccggcgggtgcacgtggtattcagggtccgcagggtccgcgtggtgataaaggtgaagcaggtgaaccgggggaaagaggattaaaagggcatcgtggttttacgggtctgcagggtctgcctggtccgcctggtccgagcggtgatcagggtgcaagcggtccggcaggtccgagcggacctcgtggacctccgggtcctgtgggtcctagtggtaaggatggggcaaatggtattcctggtcctattggtccgccgggtccgcgtgggagatcaggtgaaaccggaccggca

SEQ ID NO: 19 - DNA, 270 bp, synthetic construct
  source 1..270: mol_type = other DNA
ggtagcccaggtccagcaggtccgacaggtaaacaaggagatcgtggtgaagcaggagcacaaggaccaatgggtccaagcggtccggcaggtgcaagaggtattcaagggccgcaagggccacgcggtgataaaggggaagcaggtgaaccaggtgagagagggttaaaaggacatcgtggatttacaggactgcagggtttaccaggtccgccgggaccgagcggagatcaaggtgcaagcggtccggcgggtccgagtggtcctcgt

SEQ ID NO: 20 - DNA, 1080 bp, synthetic construct
  source 1..1080: mol_type = other DNA
ggagctaggggtatccagggtccgcagggcccgcggggagataaaggggaagcgggtgaaccgggtgaacgtggcttgaagggtcatcgtggcttcaccggcctgcagggcttgccgggtccgccgggtccgtccggtgcccgtggcatccagggaccgcaaggaccgcgtggagacaaaggtgaagcgggtgaaccgggtgagcgcggcctcaagggccaccgcggcttcaccggtttgcaaggcctgccgggtccgccgggtccgtcgggcgcgcgtggtattcaaggcccgcaaggtccacgcggcgacaaaggcgaagcgggtgagccgggcgaacgcggcctgaaaggtcaccgcggttttacgggtctgcagggcctgccgggtccaccgggtccgagcggtgctcgcggcatccagggcccgcaaggtccgcgtggtgataagggtgaggcgggtgagccgggagaacgtggtctgaagggtcatagaggcttcaccggtctgcaaggtctgccgggtccaccgggcccgagcggcgctaggggcattcagggcccgcaggggccgcgtggtgataagggcgaggccggtgagccgggcgaacgtggtttaaaaggtcatcgcggcttcacgggcctccaaggtctgcctggcccaccgggtccgagcggcgcgcgtggcattcaaggcccacagggtcctcgtggcgacaaaggcgaggcaggcgagccgggtgagcgcggtctgaaagggcaccgtggttttaccggtttgcaaggcctgccgggcccaccgggcccgtccggtgcacgtggcattcagggtccgcagggtccgcgtggcgacaagggcgaagctggtgagccgggtgagcgcggccttaagggtcaccgtggctttaccggtttacaaggtttgccgggccctccgggtccaagcggtgcacgtggtatccaaggcccacagggtccgcgtggggataaaggcgaagcgggtgaaccgggcgagcgcgggctgaagggtcaccgtggctttactggcctgcagggtctgccgggtccaccgggtccgtct

SEQ ID NO: 21 - DNA, 1083 bp, synthetic construct
  source 1..1083: mol_type = other DNA
ggatcacccgggccggcgggcccgaccggtaaacagggtgaccgcggtgaggcgggtgcgcaggggccgatgggtccgagcggtccggctggtgcgcgtggtatccagggaccgcagggcccacgcggtgataaaggcgaagcaggcgaaccgggtgagcgcggtttgaagggccaccgtggttttaccggtctgcagggcctgccgggcccgccgggtccgtcaggcgatcagggtgctagcggtccggctggcccgtccggcccgcgcggtagcccgggcccagctggcccgactggtaaacagggcgaccgtggcgaggccggtgcccaaggccctatgggcccgagcggtccggcaggcgcgcggggcatccagggtccgcaaggaccaagaggcgacaagggtgaggcaggcgagcctggcgaacgtggcctgaagggtcaccgcggttttaccggtctccaaggtctgcctggtccaccgggtccgtctggagatcaaggcgccagcggccccgcgggtccctccggtccgcgtggcagcccgggtccggcaggaccaacgggtaagcagggtgatagaggcgaagccggtgcgcagggcccgatgggtccttcgggtccagcgggggcgcgtggcattcaaggtccgcagggcccgcgtggtgacaaaggtgaggcgggcgaaccgggcgagcgcggcttgaagggtcatcgtggattcaccggtttgcaaggcctgccgggccccccgggcccgagcggtgaccaaggagctagcgggccggctggtccgtcgggcccgcgtggcagtccgggtccggcgggcccaacgggcaagcaaggagatcgtggtgaggcgggtgcgcaaggtccgatgggtccgtctggtccggcgggtgcacgcggtattcaaggtccgcaggggccgcgtggtgataaaggagaagcaggcgaaccgggtgaacgtggtctgaaaggtcatcgtggcttcaccggcctgcagggtttaccaggtccaccgggtccgtctggcgaccagggtgccagcggtccggcgggcccgtccggaccgcgttaa

SEQ ID NO: 22 - DNA, 900 bp, synthetic construct
  source 1..900: mol_type = other DNA
ggaccccaaggggcgcgtggttttccgggcaccccgggcctgccgggcgtgaagggtcaccgcggttatccggggctggatggcgcgaagggtccgcaaggtgctcgcggtttcccgggaaccccgggcctgccgggcgttaaaggtcatcgtggttatccgggtctggatggtgcgaagggcccacagggtgcgcgtggtttcccgggcacccctgggctcccgggtgtgaaaggtcacagaggttacccgggcctggatggtgccaaaggtccgcaaggtgcgcgtggttttccgggcacgccgggcttgccgggtgttaaaggtcaccgtggttatccgggtctggacggcgcaaagggtcctcagggtgcacgcggcttcccgggtacgccgggccttccgggcgtgaagggccaccgcggttatccgggtttggacggtgccaagggtccgcagggtgcgcgtggattcccgggaaccccaggcctgccaggtgtgaagggccaccgtggctacccgggcttggacggtgctaaaggaccgcagggcgcacgtggctttccgggtaccccgggcctgcctggcgtcaagggccatcgtggttacccgggcctggatggtgcaaaaggcccacagggtgcccgtggtttcccgggtactccgggcctgccgggcgtaaaaggccatcgcggttacccgggtttggatggcgctaaaggcccacaaggtgctagagggttccctggcaccccgggtctgccgggcgttaaaggccatcgtggttacccgggcttggacggcgcgaagggtccgcaaggcgcgcgtggttttccgggtaccccgggtctgccaggcgttaagggtcaccgcggttacccgggtttagacggtgcgaaa

SEQ ID NO: 23 - DNA, 972 bp, synthetic construct
  source 1..972: mol_type = other DNA
ggtgctaggggattcccgggaaccccgggtctgccaggcgtgaaaggtcaccgcggttacccgggcctcgacggcgcgaagggtgcccgtggttttccgggaaccccgggcttgccaggtgtcaagggccatcgtggttacccgggtctcgatggtgcaaagggtgcgagaggcttcccgggcaccccgggcctgccaggggtgaaaggccacagaggctatcctggcttggatggtgccaagggtgcacgtggattcccgggcactccgggtctgccgggcgtgaagggccaccgcggttatccgggcctggacggtgctaaaggcgcgcgtggttttccgggtacgccgggcttgccaggtgttaagggccaccgtggctacccggggctggatggtgccaaaggtgctcgcggtttcccgggaaccccgggtctgcctggcgtgaagggtcatcgtggttacccgggcttggacggcgctaagggtgcgcgtggttttccgggcaccccgggtctgccgggggtgaaaggtcaccgcggttatcccggtctggatggtgcgaagggtgcgcgtggcttcccgggcaccccgggcctgccgggtgttaaaggtcatcgtggttacccgggcctggatggtgccaagggcgctcgcggttttccgggcacgccaggtttaccgggggtcaaaggccatcgtggctatccgggtttagatggcgcgaaaggcgcacgcggattcccgggaaccccgggcctgcctggcgttaaaggccaccgcggttacccgggccttgacggcgcgaaaggcgcgcgtggttttccgggcaccccgggtctgccgggtgttaaaggtcaccgtggctatccgggtctggacggtgcaaaaggtgcacgtggtttcccggggactccgggcctgccgggtgttaagggccatcgtggttacccgggtttggacggtgcgaag

SEQ ID NO: 24 - DNA, 1008 bp, synthetic construct
  source 1..1008: mol_type = other DNA
ggatttcccgggactccaggtctgcccggcgtgaagggccaccgcggttatccgggcttggacggtgcgaaaggcttcccgggcacgccaggcttacctggtgtgaagggccatcgtggttacccggggctggatggtgcgaagggttttccgggcaccccgggtctgccgggcgtaaagggccaccgcggttatccgggcctggacggtgcaaaaggttttccgggcaccccgggcctgccgggtgtgaagggccaccgcggttacccgggtttggacggtgccaagggattcccaggtacgccgggtctgccgggggtgaagggccatagaggctacccgggcttggatggtgcgaaaggtttcccgggaacccctggtctgccgggcgttaagggccaccgtggttatccgggtctggatggcgcgaaaggttttccgggaaccccaggtctgccgggcgttaaaggtcatcgtggttacccggggctcgatggtgccaaaggttttccgggcaccccgggtctgccgggcgtcaaaggtcaccgcggttatccgggtctggatggtgcgaaaggcttcccgggaaccccgggcctgcctggcgtgaaaggccatcgtggttatccgggtctggatggtgctaaaggattcccgggaaccccgggcctgccgggcgttaagggtcaccgtggttacccgggcctggatggcgctaagggtttcccgggtacgcccggcctgccgggcgtcaaaggtcatcgtggttacccgggtttagacggcgcaaaaggtttcccgggaaccccgggtctgccgggtgttaagggccatcgtggctatccgggcttggacggtgcaaagggttttcctggtactccaggcttgcccggcgttaagggccaccgtggttacccgggtttggacggcgctaaaggctttccgggaaccccgggtcttccgggcgtgaaaggtcaccgtggttacccgggtctggacggcgcgaag

SEQ ID NO: 25 - DNA, 1008 bp, synthetic construct
  source 1..1008: mol_type = other DNA
ggaacacccgggctgccgggtgttaagggccatcgtggctacccgggtttggacggtgcgaaaggcacccctggcctgccgggtgtcaaaggccatcgtggttatccgggtctcgacggtgcgaagggtaccccaggactgccgggtgttaaaggtcaccgcggttacccgggtctggacggcgcaaaaggtactccaggcctgccgggcgtgaagggtcatcgtggttacccgggcctggacggcgcgaagggcaccccgggcttaccgggcgtgaaaggccatcgtggttatccggggttggatggcgcaaagggtaccccgggtctgcctggtgttaagggccaccgcggttatccgggcctggatggtgcgaaaggtacgccgggattgccgggtgtgaagggtcatcgtggttacccgggtctggatggtgccaaaggcacgccaggccttccgggcgtcaagggccaccgcggttacccgggcctggatggtgcgaaaggtaccccgggtttgcctggtgttaagggccaccgcggttatcctggtttggacggcgcgaaaggaaccccgggcctgccgggtgttaaaggccatcgtggctacccgggtctggacggcgctaaaggcactccgggtctgccgggcgtgaagggccaccgtggttatccgggtttagacggcgcgaaaggcaccccaggcctgccgggtgtcaagggtcaccgcggttacccgggtctggatggtgccaagggcaccccgggcttgccgggcgtgaaaggacatcgtggttacccgggcctggatggtgctaagggcacgccaggtcttccgggtgtgaaaggtcaccgtggttatccgggtctggacggtgcaaagggaaccccggggctgccgggcgtaaaaggccacagaggttatccgggtcttgatggtgcgaagggcaccccgggcctgccgggcgttaaaggtcaccgtggttacccgggtttggacggcgctaaa

SEQ ID NO: 26 - DNA, 936 bp, synthetic construct
  source 1..936: mol_type = other DNA
ggagaaagggggttgaagggacaccgcggttttactggtttgcaaggcctgccgggccctccgggtccgtctggcgatcagggtgcaagcggcccggcgggtccgtcgggcccgcgtggtgagcgcggtcttaagggccatcgtggtttcaccggtttacaaggtctgccgggcccgccgggtccgagcggtgatcaaggggcctccggtccggctggcccgtccggcccaagaggcgaacgtggtctgaaaggtcatcgtggattcaccggactgcagggtctgcctggtccgcctggtccgtcaggcgaccaaggtgcgagcggtccggcgggtccgtccggtccgcgtggtgaacgtggtctgaaaggccaccgcggcttcaccggtttgcaaggcctgccaggcccaccgggtccgtctggcgaccagggagccagcggtccggctggcccatctggcccacgcggcgagcgcggtctgaaaggccaccgtggctttacgggcttgcagggtctcccgggcccaccgggcccgagcggtgatcagggtgccagcggaccggcaggcccctctggtccgcgtggtgaacgtggcctgaaaggtcatcgtggttttaccggtttacagggcctgccaggtcccccgggtccgtccggcgaccagggcgcaagcggtccggctggcccgagcggtccgcgtggcgagcgcggccttaagggccacagaggcttcacgggtctgcaaggtttgccgggtccgcctggcccgtcgggcgatcagggcgcgagcggcccggcgggtccgagcggtccgcgtggcgagcgtggtctgaagggtcaccgcggttttaccggtctgcaaggtctgccgggtccgcctggcccgagcggcgaccagggagcgagcggtccggcgggtccgagtggtccgcgt

SEQ ID NO: 27 - DNA, 990 bp, synthetic construct
  source 1..990: mol_type = other DNA
ggggaaaggggactcaaaggtcaccgcggtttcacgggccttcaaggtctgccgggtcctccgggtccgagcggcgaccaaggtgcgtctggcccagcgggtgagcgtggtttaaaaggccaccgcggtttcaccggcctgcagggtttaccgggtccgcctggcccgagcggtgatcaaggtgcaagcggcccggcaggcgaacgcggtctgaaaggccatagaggttttaccggcctgcagggcttgccgggcccgccgggcccgagtggcgatcaaggtgcttccggcccggcgggtgaacgtggcctgaagggccatcgtggctttaccggtctgcagggcctgccaggtccgccgggcccgtctggcgaccagggtgcgagcggtccagccggtgagcgcggcttgaagggccaccgcggctttacgggtttgcaaggtctgcctggtccgccgggcccgtcaggcgatcaaggggcgagcggcccggcgggtgaacgtggtctgaaaggtcatcgtggattcaccggcctgcagggtctgccgggcccgcccggcccgtccggtgaccagggtgcttccggtccggctggtgagcgcggtcttaagggacaccgtggcttcaccggtctgcagggcttgccgggtcctccggggccgagcggcgaccagggtgcgtccggcccggcgggtgaacgtggtctgaagggccaccgtggttttaccggcttgcaaggtctgccgggtccaccgggtccgtctggcgatcagggagccagcggtccggcaggcgagcgtggtttgaagggtcaccgtggattcaccggcctgcaggggctgccgggcccgccgggtccgtcgggcgatcagggagcgagcggtccggcaggtgagcgtggtctgaaaggccatcgtggcttcactggtttgcaaggcctgcctggcccaccgggtccgagcggtgaccagggtgccagcggtccggct

SEQ ID NO: 28 - DNA, 972 bp, synthetic construct
  source 1..972: mol_type = other DNA
ggagaaagggggcttaagggccatcgtggcttcacgggtttgcagggcctgccgggcccaccgggtccgagcggcgatcaaggcgagcggggcctgaaaggtcatcgtggctttaccggtttgcagggcctgccgggcccaccgggaccgagcggtgatcagggggagcgtggtctgaaaggtcatcgtggttttaccggtctgcagggtttgccgggtcctccgggtccgtctggcgaccaaggtgagcgtggtctgaaaggtcatcgtggtttcaccggtttacaaggcctgccgggcccaccgggtccgagcggtgaccaaggcgagcgtggcctgaaaggccaccgcggtttcaccggtcttcagggtctgccgggcccaccgggtcccagcggtgaccagggtgaacgcggtctgaagggtcacagaggcttcaccggtctccaaggcttaccgggtccgccgggcccgagcggtgatcagggcgaacgtggtctgaagggccaccgtggctttacgggtctgcaaggcctgccgggtccgccgggtccgtcgggcgaccagggtgagcgcggtttgaagggccaccgtggattcaccggcctgcagggcctgccgggaccgcctggtccgtcaggtgatcagggtgagcgcggtctcaagggtcatcgtggttttaccggtttacaaggtttgccgggtccgcctggcccgtctggtgatcaaggcgaacgtggtctgaaaggccaccgcggcttcactggtctgcagggcttgccgggcccaccgggtccgtccggcgatcaaggcgaacgtggtctgaagggtcaccgcggattcacgggcctgcagggcctgccgggtccgccaggtccgtccggcgaccagggcgaacgcgggctgaaaggtcaccgtggctttaccggtttgcaaggactgccgggtccgccgggcccgagcggcgaccag

SEQ ID NO: 29 - DNA, 1008 bp, synthetic construct
  source 1..1008: mol_type = other DNA
ggagaaagggggctgaagggtcaccgtggttttaccggtttgcaaggtttgccgggtccgccgggtgagcgcggcctgaagggtcatcgtggcttcaccggcctgcaaggcctgccgggtccgccgggtgagcgtggtctcaagggtcatcgtggctttaccggtttacagggtctgccgggcccaccgggcgagcgcggtctgaaaggccatcgtggattcaccggcctgcagggcttgccgggtccaccgggcgaaagaggcttaaaaggtcaccgtggttttacgggtctgcaaggtctgccgggtccgccgggtgaacgtggccttaagggtcaccgcggcttcactggtctgcagggtttgccgggtccgcctggtgaaaggggtctgaaaggccatcgtggtttcaccggtctgcagggcctgccgggcccaccgggtgagagaggcttgaagggtcaccgtggttttacgggcctgcagggtctgccgggcccgccgggtgagcgcggtcttaagggccaccgtggattcaccggccttcaaggtctgccgggtccgccgggggagcgtggcctgaaaggtcatcgcggcttcaccggtttgcaaggtttgccaggcccaccgggcgaacgtggcctgaaagggcaccgtggttttactggtttgcagggcctgccgggcccgccgggcgagcgcggtttgaagggccaccgtggtttcacgggcctgcaaggtctgccgggccctccgggagaacgcggtctgaaaggtcaccgtggcttcaccggcctgcagggtctaccgggtccaccgggcgagcgcggactgaaaggtcatcgcggcttcaccggtctgcagggcctgccgggtccgccgggtgaacgtggcctgaaaggccaccgcggttttaccggtttacaaggcttgccgggtccgccgggtgaacgtggcttgaagggccatcgtggctttaccggtcttcaggggctgccgggcccgcct

SEQ ID NO: 30 - protein, 336 aa, synthetic construct; identical to SEQ ID NO: 10

SEQ ID NO: 31 - protein, 9 aa, synthetic construct
GTPGLPGVK

SEQ ID NO: 32 - protein, 12 aa, synthetic construct
GTPGLPGVKGHR

SEQ ID NO: 33 - protein, 9 aa, synthetic construct
GYPGLDGAK

SEQ ID NO: 34 - protein, 21 aa, synthetic construct
GYPGLDGAKGTPGLPGVKGHR